SEQUENCE LISTING (6 sequences)

SEQ ID NO: 1 - DNA, 19 bp, Homo sapiens
ggatgacccccctcagata

SEQ ID NO: 2 - DNA, 19 bp, Homo sapiens
cttatttaaggggaacgtg

SEQ ID NO: 3 - DNA, 18 bp, Homo sapiens
catgctaagacttcacca

SEQ ID NO: 4 - DNA, 18 bp, Homo sapiens
tcgttgaacaaacgaacc

SEQ ID NO: 5 - DNA, 21 bp, Homo sapiens
tttcggtcaccctgaagttta

SEQ ID NO: 6 - DNA, 21 bp, Homo sapiens
agcgaaggcttctcaaatcat

(19) United States
(12) Patent Application Publication
(10) Pub. No.: US 2009/0269758 A1
(43) Pub. Date: Oct. 29, 2009

(54) Diagnostic methods and kits for functional disorders

(76) Inventors: Richard G. Boles, San Marino, CA (US); Essam A. Zaki, Los Angeles, CA (US)

Correspondence Address: STAHL LAW FIRM, 2 MEADOWSWEET LANE, SAN CARLOS, CA, 94070, US

(21) Appl. No.: 12/319,541

(22) Filed: Jan. 8, 2009

Related U.S. Application Data
(63) Continuation-in-part of application No. PCT/US2008/003994, filed on Mar. 27, 2008.
(60) Provisional application No. 60/920,750, filed on Mar. 29, 2007.

(51) Int. Cl. C12Q 1/68

(52) U.S. Cl. 435/6

(57) ABSTRACT

The present invention relates to methods for the diagnosis of functional disorders in humans. A method of the invention, in certain embodiments, comprises the detection of one or more polymorphisms in mitochondrial DNA of a human. The current invention further provides kits for use in a method of the invention.

This application is a continuation-in-part of International Application PCT/US2008/03994, with an international filing date of Mar. 27, 2008, pending; which claims the benefit of U.S. Provisional Application Ser. No. 60/920,750, filed Mar. 29, 2007, now abandoned; all of which are incorporated herein by reference.

### 1.0 FIELD OF THE INVENTION

The invention relates to methods and kits for the diagnosis of functional disorders.

### 2.0 BACKGROUND

Functional disorders are various conditions that affect the well-being of a human. Examples of functional disorders are chronic fatigue syndrome (CFS), migraine, irritable bowel syndrome (IBS), depression, fibromyalgia, and complex regional pain syndrome. Symptoms of functional disorders commonly relate to nervous function (especially function of the autonomic nervous system), muscle function and/or gastrointestinal function.

Functional disorders are believed to be common, affecting the well-being and quality of life of many and thereby also having a significant economical impact. In order to alleviate the impact of functional disorders, it is necessary to diagnose those disorders and to take remedial measures. A challenge in the diagnosis of functional disorders is that a physiological or anatomical cause for functional disorders cannot be identified. Moreover, the symptoms observed in patients suffering from functional disorders are also seen in patients not suffering from functional disorders. Rather, symptoms associated with functional disorders may result from other afflictions, which may be less severe or more severe, and which may be of a physiological nature and/or a psychological nature.

Functional disorders were found to track patterns of mitochondrial lineage. Mitochondria have their own DNA in the form of a plasmid of 16569 nucleotides that is passed on through maternal inheritance. Symptoms of functional disorders are typically found in family members who are direct descendants from a maternal donor with those symptoms.

Methods and kits (reagents) to diagnose functional disorders would be highly desirable. The current invention provides such methods and kits.

### 3.0 SUMMARY OF THE INVENTION

The current invention relates to methods for the diagnosis of functional disorders. A method of the current invention, in certain embodiments, comprises the detection of a polymorphism in mitochondrial DNA of a human. In certain embodiments, a polymorphism detected with a method of the invention is an indicator that the carrier of the polymorphism is more likely to suffer from functional disorders than an individual who is not a carrier of the polymorphism.

In certain embodiments, a method of the invention detects a polymorphism at one, two, three, four, five or more nucleotides of the human mitochondrial genome. In certain embodiments, a method of the invention detects a polymorphism at one, two, three, four, five or more of nucleotides numbers 239, 2259, 3010, 4727, 4745, 7337, 9380, 13326, 13680, 14831, 14872, and/or 16519, of the human mitochondrial genome. In certain embodiments, a method of the invention detects a defined polymorphism at one, two, three, four, five or more nucleotides by detecting the presence of a defined nucleotide. In certain preferred embodiments, a method of the invention detects the presence or absence of one, two, three, four, five or more polymorphisms of 239C, 2259T, 3010A, 3010G, 4727G, 4745G, 7337A, 9380A, 13326C, 13680T, 14831A, 14872T, 16519T, and/or 16519C (each being the number of the nucleotide in the human mitochondrial genome followed by the nucleotide that is detected if the defined polymorphism is present).

In certain other embodiments, the absence of one or more polymorphisms of the current invention is an indicator that the individual who is not a carrier of such one or more polymorphisms is more likely not to suffer from functional disorders than is an individual who carries one or more of these polymorphisms.

In certain embodiments, a method of the invention detects the absence of a polymorphism at one, two, three, four, five or more nucleotides of the human mitochondrial genome. In certain embodiments, a method of the invention detects the absence of a polymorphism at one, two, three, four, five or more of nucleotides numbers 239, 2259, 3010, 4727, 4745, 7337, 9380, 13326, 13680, 14831, 14872, and/or 16519, of the human mitochondrial genome. In certain preferred embodiments, a method of the invention detects the absence of one, two, three, four, five or more of the defined polymorphisms of 239C, 2259T, 3010A, 3010G, 4727G, 4745G, 7337A, 9380A, 13326C, 13680T, 14831A, 14872T, 16519T, and/or 16519C.

In certain other embodiments, the invention provides kits for the detection of the presence or absence of a polymorphism for the diagnosis of functional disorders.

### 4.0 BRIEF DESCRIPTION OF THE DRAWINGS

FIG. 1: Odds ratio for specific phenotype versus healthy volunteer status in mtDNA haplogroup H (relative to non-H haplogroups) are shown.

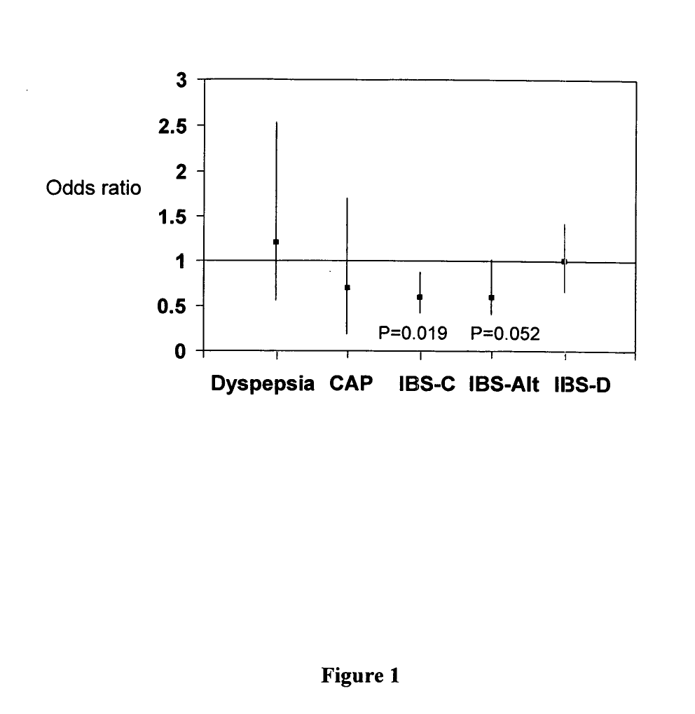

### 5.0 DETAILED DESCRIPTION OF THE INVENTION

The current invention is related to methods and kits for the diagnosis of functional disorders. In certain embodiments, the current invention relates to methods comprising the detection of one or more polymorphism and the determination of a predisposition and/or affliction with functional disorders.

Particular methods and/or compositions described herein are provided for purposes of illustration and not to limit the invention in any way. The terminology used herein is for the purpose of describing particular embodiments only, and is not intended to be limiting, since the scope of the present invention will be limited only by the appended claims. Where a range of values is provided, each intervening value between the upper and lower limits of that range, to the tenth of the unit of the lower limit, is also specifically disclosed, unless the context clearly dictates otherwise.

Unless expressly defined otherwise, each and every technical, scientific and other term used herein has the same meaning and the same breadth as commonly understood by one of ordinary skill in the art to which this invention belongs. As used herein and in the appended claims, the singular forms “a”, “and”, and “the” include plural referents unless the context clearly dictates otherwise. Although any methods and materials similar or equivalent to those described herein can be used in the practice or testing of the present invention, the preferred methods and materials are described. All publications mentioned herein are incorporated herein by reference for any purpose and to disclose and describe the methods and/or materials in connection with which the publications are cited.

### 5.1 METHODS TO DIAGNOSE FUNCTIONAL DISORDERS

The current invention relates to methods that are useful for the diagnosis of functional disorders. A method of the current invention, in certain embodiments, comprises the detection of a polymorphism in mitochondrial DNA of a human. In certain embodiments, a method of the invention comprises detecting one, two, three, four, five, or more polymorphisms in the mitochondrial DNA of a human. Background on human mitochondrial DNA is also described in U.S. Patent Application Nos. 20040029133; 20050026167; 20050123913; 20060078881; 20060147925; 20070190534; and in U.S. Pat. Nos. 5,670,320; 6,605,433; 6,759,196, all of which are incorporated herein by reference for all purposes, and a method of the current invention may be applied to a mitochondrial DNA described in any of these references.

In certain embodiments, a polymorphism of the invention is an indicator that the carrier of the polymorphism (referred to herein as “carrier”) is more likely to suffer from functional disorders than an individual who is not a carrier of the polymorphism (referred to herein as “non-carrier”). In certain embodiments, the presence of one, two, three, four, five, or more polymorphisms of the invention in a human is an indicator that the carrier of the polymorphisms is more likely to suffer from functional disorders than an individual who is not a carrier of the polymorphisms. In certain embodiments, a carrier of a polymorphism of the invention is at least 2× (two times) more likely to suffer from functional disorders than a non-carrier, or at least 3×, or at least 4×, or at least 5×, or at least 6×, or at least 7×, or at least 8×, or at least 9×, or at least 10× more likely. In certain embodiments, a carrier of one, two, three, four, five, or more polymorphisms of the invention is at least 2× (two times) more likely to suffer from functional disorders than a non-carrier of those polymorphism(s), or at least 3×, or at least 4×, or at least 5×, or at least 6×, or at least 7×, or at least 8×, or at least 9×, or at least 10× more likely.

In certain embodiments, the presence of one, two, three, four, five, or more polymorphisms of the invention in a human is an indicator that the carrier of the polymorphisms is more than 50 percent likely to suffer from functional disorders, or more than 60 percent, more than 70 percent, more than 80 percent, more than 90 percent, more than 95 percent, or that the carrier is 100 percent likely to suffer from functional disorders.

In certain other embodiments, the absence of one, two, three, four, five, or more polymorphisms of the current invention is an indicator that the individual who is not a carrier of such one or more polymorphisms is more likely not to suffer from functional disorders than to suffer from functional disorders. In certain other embodiments, the absence of one, two, three, four, five, or more polymorphisms of the current invention is an indicator that the individual who is not a carrier of such one or more polymorphisms is at least 2× (two times) less likely to suffer from functional disorders than a carrier of such one or more polymorphisms, or at least 3×, or at least 4×, or at least 5×, or at least 6×, or at least 7×, or at least 8×, or at least 9×, or at least 10× less likely.

In certain embodiments, the absence of one, two, three, four, five, or more polymorphisms of the invention in a human is an indicator that the non-carrier of the polymorphisms is more than 50 percent likely not to suffer from functional disorders, or more than 60 percent, more than 70 percent, more than 80 percent, more than 90 percent, more than 95 percent, or that the carrier is 100 percent likely not to suffer from functional disorders.

### 5.2 FUNCTIONAL DISORDERS

The current invention relates to methods to identify a predisposition for and/or an affliction with functional disorders in a human. Functional disorders are disorders that exhibit no demonstrable structural pathology while a patient suffering from functional disorders exhibits deficient nerve and/or muscle function when compared to a healthy person. Symptoms observed in functional disorders include gastrointestinal dysmotility, gas, pain, migraine, cyclic vomiting, chronic fatigue, limb pain, constipation, diarrhea, sleep apnea, frequent urination, and/or gastroesophageal reflux. Examples of functional disorders include chronic fatigue syndrome (CFS), migraine, irritable bowel syndrome (IBS), depression, fibromyalgia, complex regional pain syndrome, nonspecific abdominal pain, chronic temporal mandibular joint pain, myalgic encephalitis, chronic pelvic pain, chronic pain syndromes, interstitial urethritis, post-traumatic stress syndrome, gulf war syndrome, functional tinnitus, sudden infant death syndrome (SIDS), and hypoglycemia.

### 5.3 POLYMORPHISMS IN MITOCHONDRIAL DNA

In certain embodiments, a method of the invention detects a polymorphism at one, two, three, four, five or more nucleotides of the human mitochondrial genome. In certain embodiments, a method of the invention detects a polymorphism at one, two, three, four, five or more of nucleotides numbers 239, 2259, 3010, 4727, 4745, 7337, 9380, 13326, 13680, 14831, 14872, and/or 16519, of the human mitochondrial genome. In certain embodiments, a method of the invention detects a polymorphism at one, two, three, four, five or more of nucleotides by detecting the presence of a defined nucleotide. In certain preferred embodiments, a method of the invention detects the presence or absence of one, two, three, four, five or more defined polymorphisms of 239C, 2259T, 3010A, 3010G, 4727G, 4745G, 7337A, 9380A, 13326C, 13680T, 14831A, 14872T, 16519T, and/or 16519C (each being the number of the nucleotide in the human mitochondrial genome followed by the nucleotide that is detected if the defined polymorphism is present). In certain preferred embodiments, a method of the invention detects the presence or absence of one or two defined polymorphisms of 3010A, 3010G, 16519T, and/or 16519C.

In certain embodiments, a method of the invention detects the absence of a polymorphism at one, two, three, four, five or more nucleotides of the human mitochondrial genome. In certain embodiments, a method of the invention detects the absence of a polymorphism at one, two, three, four, five or more of nucleotides numbers 239, 2259, 3010, 4727, 4745, 7337, 9380, 13326, 13680, 14831, 14872, and/or 16519, of the human mitochondrial genome. In certain preferred embodiments, a method of the invention detects the absence of one, two, three, four, five or more polymorphisms by assaying for the absence of 239C, 2259T, 3010A, 3010G, 4727G, 4745G, 7337A, 9380A, 13326C, 13680T, 14831A, 14872T, 16519T, and/or 16519C.

### 5.4 PATIENTS

A method of the invention, in certain embodiments, may be used on an individual who has never experienced symptoms associated with functional disorders and on an individual who has experienced such symptoms. In certain embodiments, a method of the invention may be used on an individual who has experienced symptoms associated with functional disorders on more than one occasion (i.e., who has a history of functional symptomatology). In certain other embodiments, a method of the invention may be used on an adult (at least 18 years old), a child, a male, and/or a female patient. In certain embodiments, a method of the invention may be used on an individual who has been diagnosed with one or more functional disorders, for example, chronic fatigue syndrome (CFS), migraine, irritable bowel syndrome (IBS), depression, fibromyalgia, complex regional pain syndrome, nonspecific abdominal pain, chronic temporal mandibular joint pain, myalgic encephalitis, chronic pelvic pain, chronic pain syndromes, interstitial urethritis, post-traumatic stress syndrome, gulf war syndrome, and/or functional tinnitus.

In certain embodiments, a polymorphism of the invention is detected in mitochondrial DNA of any haplogroup, sub-haplogroup and/or any haplotype. As used in the art and herein, a haplotype is a particular combination of genetic markers, many haplogroups are divided into two or more sub-haplogroups, and haplogroups and sub-haplogroups are groups of haplotypes in association with one another. Examples of known haplogroups are A, B, C, D, E, F, G, H, HV, I, J, K, L1, L2, L3, M, N, P, Q, R, T, U, V, W, X, Y, and/or Z. Background on haplogroups and haplotypes of mitochondrial DNA is also described in U.S. Patent Application Nos. 20040029133; 20050123913; and in U.S. Pat. Nos. 5,670,320; 6,759,196, all of which are incorporated herein by reference for all purposes.

### 5.5 DETECTION OF POLYMORPHISMS

A polymorphism of invention may be detected using any method known in the art. For example, any method capable of determining the sequence of a polynucleotide or oligonucleotide may be used to identify the nucleotide at the site of the nucleotide number of the polymorphism that is the subject of analysis. A method useful for the detection of a polymorphism of the invention, in certain embodiments, may sequence mitochondrial DNA, or a DNA or RNA copy thereof, at and/or around the site of the polymorphism. In certain other embodiments, a method useful for the detection of a polymorphism of the invention may detect a sequence at and/or around the site of a polymorphism, for example, through restriction enzyme digestion. In certain other embodiments, a method useful for the detection of a polymorphism of the invention may examine properties of a DNA or RNA oligonucleotide or polynucleotide, for example, migration properties on a polyacrylamide gel. In certain other embodiments, a method useful for the detection of a polymorphism of the invention may examine the amplification (in other words, multiplication) of a polynucleotide or oligonucleotide sequence through the polymerase chain reaction or another technique involving an amplification of a polynucleotide or oligonucleotide sequence.

In certain embodiments, a polymorphism of the invention is detected by analyzing mitochondrial DNA from a sample obtained from a human. Examples of such a sample include blood, skin, saliva, cerebrospinal fluid, muscle tissue, nerve tissue, placenta, and any other type of organ, tissue and/or cell. Mitochondrial DNA for analysis of the presence of a polymorphism of the invention may also be obtained, for example, by growing cells in culture and by obtaining mitochondrial DNA from such cells.

Examples of methods known in the art that are useful for detecting a polymorphism in mitochondrial DNA include, but are not limited to, any direct sequencing methodology such as cyclosequencing, as well as any indirect sequencing method, of which examples include fluorescence in-situ hybridization (FISH), Southern blot analysis, single stranded conformation analysis (SSCP), denaturing gradient gel electrophoresis, denaturing high pressure liquid chromatography (DHPLC), RNAase protection assays, allele-specific oligonucleotides (ASO), dot blot analysis, PCR-SSCP, allele-specific PCR, cleavase fragment length polymorphism (CFLP), temperature modulation heteroduplex chromatography (TMHC), sandwich hybridization methods,

A polymorphism of the invention may also be detected, for example, by differential hybridization techniques using allele-specific oligonucleotides. For example, a polymorphism may be detected on the basis of the higher thermal stability of the perfectly matched probes as compared to the mismatched probes.

In certain embodiments, a hybridization reaction in any of the methods for detecting a polymorphism of the invention may be carried out in any format, for example, a filter-based format, Southern blots, slot blots, “reverse” dot blots, solution hybridization, solid support hybridization, solid support based sandwich hybridization, bead-based hybridization, array-based hybridization, chip-based hybridization, silicon chip-based hybridization, and microtiter well-based hybridization formats.

In certain embodiments, a hybridization reaction in any of the methods for detecting a polymorphism of the invention may be carried out using a solid support of any kind, for example, supports comprising organic and/or inorganic polymers, porous supports, small beads, natural and/or synthetic supports, for example, nitrocellulose, nylon, glass, quartz, diazotized membranes (paper or nylon), silicones, polyformaldehyde, cellulose, and cellulose acetate. Or, for example, plastics such as polyethylene, polypropylene, polystyrene, and the like can be used as solid supports in certain embodiments. Or, for example, paper, ceramics, metals, metalloids, semiconductive materials, cermets or the like can be used as solid supports in certain embodiments. In certain other embodiments, substances that form gels can be used, for example, proteins (e.g., gelatins), lipopolysaccharides, silicates, agarose and polyacrylamides. In certain other embodiments, a hybridization reaction in any of the methods for detecting a polymorphism of the invention may be carried out in the liquid state, such as denaturing high pressure liquid chromatography (DHPLC). In certain other embodiments, electronic chips may be employed to obtain direct or indirect sequence information.

In certain embodiments, an amplification-based assay is used to detect a polymorphism. In such amplification-based assays, mitochondrial polynucleotide sequences act as a template in an amplification reaction (e.g., PCR). In certain other embodiments, a suitable amplification method may be a ligase chain reaction (LCR), a transcription amplification, or a self-sustained sequence replication.

Background on detecting a polymorphism, including methods and reagents used in those methods, are also described in U.S. Patent Application Nos. 20070254289; 20070277251; 20080015112; and in U.S. Pat. Nos. 5,976,798; 6,087,107; 6,268,142; 6,284,466; 6,322,978; 6,355,425; 6,379,671; 6,403,307; 6,485,908; 6,492,115; 6,551,780; 6,689,561; 6,713,258; 6,746,839; 7,033,753; 7,105,299; 7,169,561; 7,273,699; 7,316,903; 7,332,277, all of which are incorporated herein by reference for all purposes, and methods, reagents, equipment and anything else useful for detecting a polymorphism described in these references may be used to detect a polymorphism of the current invention.

### 5.6 KITS

The current invention also provides kits that are useful for some or all steps of a method of the invention. In certain embodiments, a kit of the invention is useful for some or all steps in the detection of the presence of a polymorphism of the invention. In certain other embodiments, a kit of the invention is useful for some or all steps for in detection of the absence of a polymorphism of the invention.

A kit of the current invention in certain embodiments comprises reagents useful in detecting a polymorphism of the invention. In certain embodiments, a kit of the invention comprises instructions for using the kit in detecting a polymorphism of the invention. In certain embodiments, a kit of the invention comprises instructions for determining whether a person is likely to suffer from functional disorders.

A kit of the current invention in certain embodiments comprises an oligonucleotide complementary to mitochondrial DNA 5′ to a site of a polymorphism (nucleotide number where the polymorphism is located) of the invention, or 3′ to a site of a polymorphism of the invention. In certain embodiments, a kit of the invention comprises an oligonucleotide complementary to mitochondrial DNA around the site of a polymorphism of the invention, for example, so that the last, or second to last, or third to last nucleotide at the 3′ end of the oligonucleotide is at the site of the polymorphism. An oligonucleotide in a kit of the invention may be 4 to 50 nucleotides in length, or 6 to 40 nucleotides, or 8 to 30 nucleotides, or 10 to 24 nucleotides, or 12 to 20 nucleotides.

In certain embodiments, a kit of the invention comprises two oligonucleotides complementary to mitochondrial DNA, where one oligonucleotide is complementary 5′ to a site of a polymorphism of the invention and the other oligonucleotide is complementary 3′ to the site of the polymorphism. In certain embodiments, a kit of the invention comprises two oligonucleotides complementary to mitochondrial DNA at two sites that are separated by 0 to 2000 nucleotides, or 0 to 1000 nucleotides, or 0 to 500 nucleotides, or 0 to 200 nucleotides, or 50 to 400 nucleotides. In certain embodiments, a kit of the invention comprises two oligonucleotides that are complementary to sequences that are in the same strand of mitochondrial DNA or to sequences that are in opposite strands of mitochondrial DNA. In certain other embodiments, a kit of the invention comprises an array of oligonucleotides, for example, an array of oligonucleotides on a solid support.

In certain other embodiments, an oligonucleotide in a kit of the invention is degenerate (and therefore capable of specifically to more than one sequence). In certain other embodiments, an oligonucleotide of a kit of the invention is labeled (for example, to facilitate detection of the oligonucleotide or a compound or complex comprising the oligonucleotide). An oligonucleotide of a kit of the invention may be labeled, in certain embodiments, with a radiolabel, an enzyme, a fluorescent compound, streptavidin, avidin, biotin, a magnetic moiety, a metal-binding moiety, an antigen, an antibody, and/or an antibody fragment.

In certain other embodiments, a kit of the invention comprises an enzyme capable of catalyzing a step in a diagnostic method for which the kit is useful, for example, a kit may comprise a polymerase, a DNA polymerase, an RNA polymerase, a heat stable polymerase, a PCR polymerase, a ligase, a kinase, a restriction enzyme. In certain embodiments, a kit of the invention comprises a buffer, a buffer useful for an enzyme in the kit, a nucleotide, a triphosphate nucleotide (for example, ATP, CTP, GTP and/or TTP), a tube, an Eppendorf tube, a filter, a filter-paper, or any other component or reagent useful in a method of the current invention.

In certain other embodiments, a kit of the invention comprises instructions for using the kit in the diagnosis of functional disorders.

Background on components, reagents and instructions useful for a kit of the invention is also described in U.S. Patent Application Nos. 20070254289; 20070277251; 20080015112; and in U.S. Pat. Nos. 5,976,798; 6,087,107; 6,268,142; 6,284,466; 6,322,978; 6,355,425; 6,379,671; 6,403,307; 6,485,908; 6,492,115; 6,551,780; 6,689,561; 6,713,258; 6,746,839; 7,033,753; 7,105,299; 7,169,561; 7,273,699; 7,316,903; 7,332,277, all of which are incorporated herein by reference for all purposes, and components, reagents and instructions described in these references may be used in a kit of the current invention.

The present invention is further illustrated by the following examples, which are not intended to be limiting in any way whatsoever.

### EXAMPLES

### Example 1

### Two Mitochondrial DNA Polymorphisms Are Associated With Migraine Headache and Cyclic Vomiting Syndrome

1.1 Abstract

Mitochondrial dysfunction is a hypothesized component in the multi-factorial pathogenesis of migraine without aura (MoA “common migraine”) and its variant, cyclic vomiting syndrome (CVS). In this study, the entire mitochondrial genome was sequenced in 20 haplogroup-H CVS patients, a subject group studied because of greater genotypic and phenotypic homogeneity. Sequences were compared against haplogroup-H controls. Polymorphisms of interest were tested in 10 additional CVS subjects and in 112 haplogroup-H adults with MoA. The 16519C>T polymorphism was found to be highly disease associated: 21/30 CVS subjects (70%, odds ratio=6.2) and 58/112 migraineurs (52%, odds ratio=3.6) versus 63/231 controls (27%). A second polymorphism, 3010G>A was found to be highly disease associated in those subjects with 16519T: 6/21 CVS subjects (29%, odds ratio 17) and 15/58 migraineurs (26%, odds ratio 15) versus 1/63 controls (1.6%). Our data suggest that these polymorphisms constitute a substantial proportion of the genetic factor in migraine pathogenesis, and strengthen the hypothesis that there is a component of mitochondrial dysfunction in migraine.

1.2 Introduction

Migraine is a very common condition, affecting approximately 15% of adults (1, 2), with high economic costs, especially in terms of lost time from employment and as a frequent cause of health care utilization. Although comprised of many variants, the majority of migraineurs have migraine without aura (MoA, previously known as “common migraine”). Like most other common disorders, the etiology of migraine and its variants is multi-factorial, with many known genetic components, including genes for calcium and sodium channels, dopamine and insulin receptors, Na⁺/K⁺ ATPase pump subunits, and components of mitochondrial energy metabolism (3, 4).

A mitochondrial component to migraine has been postulated. This is supported by the findings in migraine sufferers of lactic acidosis (5, 6), mitochondrial accumulations and cytochrome-c-oxidase negative fibers in skeletal muscle (5), decreased respiratory chain complex activities (5, 7), and reduced in vivo brain phosphocreatine to inorganic phosphate ratio by ³¹P magnetic resonance spectroscopy (5). In addition, co-enzyme Q10 and riboflavin, a component and a precursor of a component of the mitochondrial respiratory chain, have shown efficacy in migraine prophylaxis in double-blind, placebo-controlled clinical trials (8, 9). The mitochondrial dysfunction hypothesis of migraine was recently reviewed (10).

Mitochondria are cytoplasmic organelles that produce the bulk of the ATP for cellular energy needs. Mitochondrial proteins are encoded on both the nuclear DNA (chromosomes) as well as the 16-kilobase mitochondrial DNA (mtDNA). Thus, sequence variants (polymorphisms) that adversely affect energy metabolism and predispose towards migraine pathogenesis theoretically could be on either or both of those genomes. The cytoplasmic-located mtDNA generally is derived solely from the ova without recombination, and individuals related through women carry an identical mtDNA sequence in the absence of a recent mutation.

Pilot studies have suggested a preferential maternal bias in migraine inheritance (11, 12), suggesting the presence of disease-predisposing mtDNA sequence variants. In support of this, about 20 different mtDNA sequence variants have been associated or possibly associated with migraine (10), especially 3243A>G (13). Most of those sequence variants are located in the “coding regions” that comprise 94% of the mtDNA and that code for subunits of the respiratory chain or the transfer and ribosomal RNA molecules needed to translate those subunits. In one study (14), migraine and its variant cyclic vomiting syndrome were found to be associated with any sequence variation in a 150 base-pair area of the control region believed to regulate mtDNA replication. Furthermore, an entire mtDNA haplogroup (U) was found to predispose towards migraine with occipital stroke (15).

However, migraine is a heterogeneous phenotype in which diagnostic criteria exist but are not always definitive. As a model disorder in which to study migraine genetics, we chose cyclic vomiting syndrome (CVS), a condition that's presence or absence is almost always clear by expert application of diagnostic criteria (16). CVS is a disabling condition characterized by recurrent, distinct episodes of nausea, vomiting and lethargy separated by asymptomatic intervals (16-18). Most sufferers encounter recurrent identical episodes that often result in frequent school or work absences and multiple hospitalizations for dehydration. CVS is not rare; it was reported in nearly 2% of Western Australian (19) and Scottish (20) school children. CVS is widely believed to be a “migraine-like condition” secondary to a strong family history of migraine headache, as well as frequent prodromic symptoms, headache, abdominal pain, evolution to migraine headache, and a positive response to “anti-migraine” medications in CVS patients (21, 22). mtDNA sequence variation is hypothesized to be a substantial risk factor in the pathogenesis of CVS due to the preferential maternal inheritance of functional disorders, including MoA, and the presence of an energy-depleted pattern on urine organic acid measurements in most cases (17). In addition, lactic acidosis, reduced electron transport chain activities and/or heteroplasmic mtDNA sequence variants have been reported in selected cases (23-25). Most mtDNA sequence variants in CVS patients have been reported in the 1 kb mtDNA control region (14, 26), although 3243A>G and large rearrangements have been reported in the 15.6 kb comprising the coding regions (26-30).

In this study, the entire mtDNA was sequenced in 20 individuals with CVS. Any potential disease-associated sequence variants were assayed in an additional 10 CVS cases and in 112 adults with MoA. In order to minimize background mtDNA sequence variability (noise), all patient and control subjects belonged to mtDNA haplogroup H.

1.3 Subjects and Methods

1.3.1 Subjects

The CVS subjects of whom the entire mtDNA was sequenced included 20 individuals from an earlier study (17) who were ascertained randomly throughout North America based upon postal codes from the database of the Cyclic Vomiting Syndrome Association (CVSA). Additional CVS subjects who were only assayed for selected mtDNA polymorphisms included six subjects recruited by the above means, and four subjects recruited as part of another earlier study on CVS with co-morbid neuromuscular disease (24). All subjects were unrelated, met the research definition for CVS (16) as determined by telephone interview, and belong to mtDNA haplogroup H.

The adult migraineur group consisted of 77 hospital-based patients from a headache clinic near Frankfurt and 35 outpatients recruited from an outpatient clinic in Munich, both from Germany. All subjects were unrelated, met the International Headache Society definition of migraine without aura (31) as determined by a mailed-in questionnaire (32) and belong to mtDNA haplogroup H. A telephone interview was performed in cases with diagnostic uncertainty.

The control group consisted of 195 full-mtDNA sequences from published sources, 213 additional almost-complete mtDNA sequences (missing the control region), and 36 healthy Caucasian children ascertained in Los Angeles who were genotyped for the polymorphisms of interest (Table 1). Only haplogroup H sequences from individuals ascertained as part of a population or control study from Europe or North America were included. To reduce potential bias, we excluded samples ascertained due to any illness or symptoms, from self-selected groups (commercial heritage testing), and from islands with small founding and/or geographically-isolated populations (Iceland and Sardinia).

Informed consent was obtained from each subject or parent, except for de-identified control subjects, and all aspects of the study was approved by the Childrens Hospital Los Angeles Institutional Review Board.

TABLE 1
Control Haplogroup H mtDNA Sequences

| | | | Number | Number | |
|---|---|---|---|---|---|
| | Number of | | with | with | |
| Subject Population | Subjects | Area Sequenced | 16519T | 3010A | Reference |
| American and British | 213 | Coding regions only | N/A | 69 | 33 |
| European-American | 103 | Entire mtDNA | 24 | 33 | 34 |
| European-American | 36 | 16519 and 3010 only | 11 | 14 | * |
| European-American | 7 | Entire mtDNA | 4 | 1 | 35 |
| Italian | 54 | Entire mtDNA | 15 | 12 | 36 |
| Finnish | 31 | Entire mtDNA | 9 | 14 | 37 |

* The present study

1.3.2 Methods

DNA was isolated from blood by standard methods or from saliva using a commercially-available kit (Oragene, DNA Genotek Inc., Ottawa, ON, Canada). Haplogroup H was defined in the conventional manner as the presence of a C at position 7028, as listed in published sequence databanks, by cyclosequencing or by PCR/restriction fragment length polymorphism (RFLP; 16519: HaeIII F GGATGACCCCCCTCAGATA (SEQ ID NO:1), R CTTATTTAAGGGGAACGTG (SEQ ID NO:2); 3010: BccI F CATGCTAAGACTTCACCA (SEQ ID NO:3), R TCGTTGAACAAACGAACC (SEQ ID NO:4)).

The entire mtDNA was amplified using 28 overlapping primer sets (26, 14; and additional sets available upon request). Cyclosequencing was performed by SeqWright (Houston, Tex.), Agencourt (Beverly, Mass.), or Eton (San Diego, Calif.). Individual sequences were aligned and compared on Sequencher® software (Gene Codes Corp., Ann Arbor, Mich.) versus our reference sequence. Our reference consists of the most common nucleotide among haplogroup H individuals in our control group for each nucleotide position throughout the mtDNA, and is termed the haplogroup H reference sequence (HhRS). The HhRS is identical to the revised Cambridge Reference Sequence (rCRS) [MITOMAP] (38), which corresponds to the actual sequence of one individual of sub-haplogroup H2, with the exception of 9 changes correcting for rare and uncommon polymorphisms in the rCRS (see Table 2 legend).

Statistics were performed by WinSTAT Statistics for Windows, Kalmia Co. Inc., Cambridge, Mass., and by custom-made software.

TABLE 2
Complete mtDNA Genomic Sequence Data in 20 CVS Subjects

| Subject | mtDNA Sequences Changes Relatives to the Haplogroup H Reference Sequence |
|---|---|
| Number | (HhRS)¹ |
| 1 | 185G > A, 302.1ins, 513G > A, 3010G > A, 5460G > A, 8251G > A, 15936A > G, |
| | 16148C > T, 16519C > T |
| 2 | 152T > C, 302.1ins, 302.2ins, 2259C > T, 4745A > G, 7337G > A, 13326T > C, |
| | 13680C > T, 14206A > G, 14831G > A, 14872C > T, 16519C > T |
| 3 | 302.1ins, 302.2ins, 989T > A, 3010G > A, 6272A > G, 14869G > A |
| 4 | 302.1ins, 2259C > T, 4745A > G, 7337G > A, 7830G > A, 13326T > C, 13680C > T, |
| | 14872C > T, 16519C > T |
| 5 | 152T > C, 1438G > A, 8598T > C, 16129G > A, 16311T > C, 16519C > T |
| 6 | 3992C > T, 4024A > G, 5004T > C, 7049A > G, 8269G > A, 9123G > A, 10124T > C, |
| | 13708G > A, 14365C > T, 14582A > G, 14956T > C, 16519C > T |
| 7 | 980T > C, 3010G > A, 8020G > A, 15394T > C, 16519C > T |
| 8 | 302.1ins, 302.2ins, 951G > A, 1438G > A, 2487.1ins, 3933A > G, 4769G > A, |
| | 16261C > T, 16354C > T, 16519C > T |
| 9 | 477T > C, 3010G > A, 9091A > G, 15734G > A |
| 10 | 152T > C, 302.1ins, 709G > A, 2259C > T, 2557C > T, 4745A > G, 7337G > A, |
| | 13326T > C, 13680C > T, 14831G > A, 14872C > T, 16519C > T |
| 11 | 456C > T, 4336T > C, 10325G > A, 11719G > A, 15833C > T, 16304T > C, 16519C > T |
| 12 | 3010G > A, 16519C > T |
| 13 | 239T > C, 302.1ins, 4727A > G, 8653A > G, 9380G > A, 16311T > C, 16519C > T |
| 14 | 302.1ins, 456C > T, 6425T > C, 16304T > C, 16519C > T |
| 15 | 73A > G, 302.1ins, 11788C > T, 16240A > G, 16519C > T |
| 16 | 477T > C, 3010G > A, 9150A > G, 9380G > A, 16263T > C, 16519C > T |
| 17 | 239T > C, 302.1ins, 302.2ins, 3915G > A, 4727A > G, 9380G > A, 9836T > C, |
| | 11253T > C, 16362T > C, 16482A > G, 16519C > T |
| 18 | 73A > G, 3010G > A, 12280A > G, 16051A > G, 16162A > G, 16304T > C, 16519C > T |
| 19 | 302.1ins, 13830T > C, 14470T > A, 16093T > C, 16221C > T |
| 20 | 3335T > C, 3358G > A, 4793A > G, 5348C > T, 16519C > T |

¹The HhRS is identical to the revised Cambridge Reference Sequence (rCRS) (38), with the exception of 7 rare polymorphisms (263A > G, 310C > T, 750A > G, 1438A > G, 4769A > G, 8860A > G, 15326A > G) and two variants (309C > T and 16519T > C).

1.4 Results

All sequence variants relative to the HhRS in our 20 fully-sequenced CVS subjects are listed in Table 2. Excluding insertions in the ultra-variable 302-315 region, 20 different mtDNA variants were identified in two or more CVS subjects (Table 3), all of which are single-nucleotide polymorphisms (SNPs) listed on MITOMAP (38). Most prominently, one of the SNPs, 16519C>T, was found to be highly associated with CVS versus controls, and another, 3010G>A, was found to be highly associated with CVS in subjects with 16519C>T versus in the controls with 16519C>T (Table 4).

TABLE 3
mtDNA Single-Nucleotide Polymorphisms (SNPs) Found in 10% or More
of the 20 Fully-Sequenced CVS or 195 Fully-Sequenced Control Subjects

| | | | | | CVS Subject |
|---|---|---|---|---|---|
| | | Number with | Number with this | | Numbers |
| | mtDNA | this SNP among | SNP among 195 | | With This |
| SNP | Gene¹ | 20 CVS Subjects | Controls | P | SNP² |
| 73A > G | MT-CR | 2 | 26 | NS | 15, 18 |
| 152T > C | MT-CR | 3 | 39 | NS | 2, 5, 10 |
| 239T > C | MT-CR | 2 | 2 | NS | 13, 17 |
| 456C > T | MT-CR | 2 | 23 | NS | 11, 14 |
| 477T > C | MT-CR | 2 | 14 | NS | 9, 16 |
| 1438G > A | MT-RNR1 | 2 | 7 | NS | 5, 8 |
| 2259C > T | MT-RNR2 | 3 | 7 | NS | 2, 4, 10 |
| 3010G > A | MT-RNR2 | 7 | 61 | NS | 1, 3, 7, 9, 12, |
| | | | | | 16, 18 |
| 4727A > G | MT-ND2 | 2 | 1 | 0.02 | 13, 17 |
| 4745A > G | MT-ND2 | 3 | 7 | NS | 2, 4, 10 |
| 6776T > C | MT-CO1 | 0 | 26 | NS | — |
| 7337G > A | MT-CO1 | 3 | 3 | NS | 2, 4, 10 |
| 9380G > A | MT-CO3 | 3 | 3 | NS | 13, 16, 17 |
| 13326T > C | MT-ND5 | 3 | 3 | NS | 2, 4, 10 |
| 13680C > T | MT-ND5 | 3 | 7 | NS | 2, 4, 10 |
| 14831G > A³ | MT-CYB | 2 | 2 | NS | 2, 10 |
| 14872C > T | MT-CYB | 3 | 7 | NS | 2, 4, 10 |
| 16304T > C | MT-CR | 3 | 20 | NS | 11, 14, 18 |
| 16311T > C | MT-CR | 2 | 10 | NS | 5, 13 |
| 16519C > T | MT-CR | 17 | 53 | 1.5 × 10−7 | 1, 2, 4, 5, 6, 7, |
| | | | | | 8, 10, 11, 12, |
| | | | | | 13, 14, 15, 16, |
| | | | | | 17, 18, 20 |

¹MITOMAP: A Human Mitochondrial Genome Database (38).
²Referring to the subject numbers in Table 2.
³Amino acid change of Ala to Thr, all other SNPs do not change the amino acid sequence.
In addition, in the 302-315 region, 60% of CVS subjects had 8 or 9 Cs (versus the more-common 7 Cs) prior to the T, versus 41% of controls (P = 0.08).

TABLE 4
Prevalence of 16519T and 3010A in Cyclic Vomiting Syndrome and Migraine without
Aura

| | Cyclic | Odds | P | Migraine | Odds | P | |
|---|---|---|---|---|---|---|---|
| | Vomiting | Ratio | vs. | Without | Ratio¹ | vs. | |
| Polymorphism | Syndrome | (95% C.I.) | Control | Aura | (95% C.I.) | Control | Controls |
| 16519T | 21/30¹ | 6.2² | 2 × 10⁻⁶ | 58/112 | 3.6² | 8 × 10⁻⁶ | 63/231 |
| | 70% | (2.7-14) | | 52% | (2.2-5.9) | | 27% |
| 3010A | 9/30 | N/A | NS | 37/112 | N/A | NS | 143/444 |
| | 30% | | | 33% | | | 32% |
| 3010A | 6/21 | 17² | 8 × 10⁻⁴ | 15/58 | 15³ | 8 × 10⁻⁵ | 1/63 |
| among | 29% | (2.0-156) | | 26% | (1.9-117) | | 1.6% |
| patients with | | | | | | | |
| 16519T | | | | | | | |

C.I. = confidence interval.
¹One subject had 16519C/T heteroplasmy (both variants present) as confirmed by both sequencing and RFLP. The genotype assigned was the dominant species, T.
²Assumes a population prevalence of 2% for CVS (19, 20) and 15% for migraine (1, 2).
³The odds ratio would be higher, but to avoid dividing by zero we assumed that no controls suffer from migraine.

Excluding 16519C>T and 3010G>A, there was a mean of 5.2 mtDNA SNPs per individual among the fully-sequenced 20 CVS subjects and 4.9 mtDNA SNPs per individual among the 195 fully-sequenced controls (P=NS).

The 16519C>T polymorphism was found to be highly associated with MoA (Table 4). In addition, among the subset with 16519T, the 3010G>A polymorphism was found to be highly associated with MoA.

1.5 Discussion

Complex multifactorial conditions, usually influenced by multiple genetic and environmental factors, are the cause of the vast majority of human disease. These conditions are becoming better understood as (nuclear) genetic polymorphisms that confer an increased risk toward disease pathogenesis are rapidly being identified. Although the mitochondrial genome is small, at high copy number it constitutes up to one third of the total cellular mass of DNA and has a very-high polymorphic density (39). Thus, mtDNA sequence variation is likely to affect an individual's risk towards the development of some multifactorial conditions in a manner analogous to nuclear DNA polymorphisms (40). Migraine (including MoA and CVS) may follow such a hypothesis since a genetic component in its pathogenesis, preferential maternal inheritance and mitochondrial dysfunction are all well established. Furthermore, in converse, migraine is very common in patients with maternally inherited mitochondrial dysfunction (41). Establishing disease-associated mtDNA sequence variant(s) in migraine, or in any other condition, indicates that energy metabolism is a factor in disease pathogenesis, since the 37 mtDNA genes are exclusively involved in oxidative phosphorylation (39). In addition, disease-associated mtDNA sequence variation constitutes an important justification for the use of mitochondrial-directed therapies, some of which have demonstrated efficacy by double-blind clinical trials in migraine, and by retrospective studies and anecdotal observation in CVS (25, 42).

In the present study, we demonstrate that the common mtDNA polymorphisms 16519T and 3010A are highly associated with the most common form of migraine and the migraine-variant cyclic vomiting syndrome. Given the lack of significant disability in most MoA cases, we assume that our control group, ascertained for forensic or evolutionary studies, contains about the same proportion of migraineurs as does the general population. With this assumption, the 16519T polymorphism alone was found to have an odds ratio of 3.6 in MoA and 6.2 for CVS (Table 4). This corresponds to predicted prevalence rates of 28% and 11% for migraine in individuals with 16519T and 16519C, respectively. Even more striking is the combined effects of the two polymorphisms, which are very rarely seen together in control populations. Although the 3010 polymorphism alone does not appear to confer risk to developing migraine, on a 16519T background, the 3010A polymorphism has an odds ratio of 15 for MoA and 17 for CVS. Thus, the data predict that migraine is present in 74% of individuals with the combined 16519T/3010A genotype, and that these mtDNA polymorphisms are likely acting synergistically.

In addition, our data suggest that there may be other mtDNA polymorphic modifiers on a 16519T background, but the numbers are too small for significance in most cases (Table 3). In one case that did reach statistical significance, the 4727A>G polymorphism was found in 2/20 CVS cases versus in 1/195 fully-sequenced controls, respectively. Furthermore, three CVS subjects with 16519T but without 3010A>G, share six other polymorphisms (2259C>T, 4745A>G, 7337G>A, 13326T>C, 13680C>T and 14872C>T) in common, a combination not present in any of the 195 fully-sequenced controls (P=7×10⁴). Those three subjects are not closely related as their mtDNA sequences vary at other polymorphisms. Additional CVS samples were studied following the completion of the present manuscript (see Table 8 below).

The overall genetic component to migraine pathogenesis is moderate, with first-degree relatives being at about a two-fold increased risk for being affected themselves (43). Thus, 16519T and 3010A likely constitute a substantial proportion of the overall genetic factor in MoA. As we have full mtDNA sequences in 20 of our CVS subjects, in CVS the data do not support that these variants are in linkage disequilibrium with other deleterious mtDNA sequences (Tables 2 and 3). More likely, it is the 16519T and 3010A nucleotides themselves that affect energy metabolism in a manner that predisposes towards the development of MoA and CVS. Furthermore, excluding 16519T and 3010A, all other mtDNA SNPs were found at essentially the same frequency in our CVS subjects and controls. While we certainly cannot state that one or more of these other polymorphisms among our CVS subjects may be disease-predisposing or modifying, it does not appear that any of them are singularly important in a sizable proportion of CVS patients, at least not in haplogroup H.

The 16519T polymorphism is located in the 1-kb non-coding mtDNA control region (often referred to as the “D-loop”), not far from the origin of heavy-strand replication and putative membrane-attachment site (38). This is a very atypical mtDNA nucleotide. In terms of nomenclature, 16519T is one of nine uncommon or rare single nucleotide polymorphisms that are present in the single individual whose mtDNA was first sequenced and thus comprises the established reference sequence (rCRS) (38). Thus, while 16519C is the ancestral (present in chimpanzees) and the most common nucleotide among humans in general, 16519T is the rCRS nucleotide. While located in an area of the control region with relatively low sequence heterogeneity, 16519T is considered to have one of the highest mutation rates of any mtDNA position and has arisen multiple times in human evolution, including among Americans of West Eurasian (44), East Asian (45) African (46), and Hispanic/Native American (47) origins. Interestingly, 16519T was recently shown to be associated with diabetes and a poorer prognosis in individuals with pancreatic cancer (48). A physiological effect of this polymorphism is also suggested by its complex effects on human exercise physiology (49).

The 3010A polymorphism is located in the 16S-ribosomal RNA gene, whereas it rebuilds a base pair in a stem of the ribosomal A-site (38). Bacterial mutations in this stem confer resistance to certain antibiotics such as chloramphenicol. This nucleotide demonstrates evolutionary conservation in primates, although A is the nucleotide in mice and frogs. 3010A has appeared in human evolution at least 15 times on 10 different mtDNA haplogroups, and may be under positive selection in humans. It defines the sub-haplogroups/clusters of H1, J1, U3 (West Eurasian), D4, C (East Asian) and L2a (African) (38). J1 and D4 are overrepresented in centenarians (50), but 3010A by itself has not previously found to be statistically associated with human disease.

The mtDNA haplogroups denote sets of ancient matrilineal ancestry tens of thousands of years old. The West Eurasian haplogroup H is well suited for genetic association studies due to the relative lack of intra-group sequence variability and high prevalence. Among our fully-sequenced control group of 195 European-derived individuals with haplogroup H, the mean number of nucleotide changes throughout the entire mtDNA genome relative to the HhRS is only about 8 nucleotides (data to be published separately), versus several times this number for unrelated individuals of mixed haplogroups. Thus, limiting our study to subjects with haplogroup H substantially decreases background sequence variability and correspondingly increases statistical power. It is also practical as haplogroup H is the most common among European-derived populations, including prevalence rates of about 45% in the native population of Germanic countries, and in about 33% of North Americans of apparent-European ancestry (40). Thus, we chose to sacrifice a larger number of subjects for substantially greater genotypic homogeneity by limiting this study to subjects of haplogroup H. Unlike some haplogroups, the 16519 and 3010 nucleotide positions are quite heterogeneous among individuals within haplogroup H. For example, had we chosen to study haplogroups J or T, whereas 3010A and 16519C are nearly universal, respectively, we would have missed the association with either polymorphism. Of course, this reasoning states that we may have missed migraine-associated polymorphisms that are not heterogeneous among haplogroup H individuals, and research in individuals with other haplogroups is needed.

One potential problem with our study is the lack of a German control group. However, the polymorphic frequency of 16519T appears to be highly homogeneous among continental European-derived haplogroup H populations, including 27% from the USA, 28% from Italy, and 29% from Finland (Table 1). Also considering that the migraineurs were ascertained from two large cities in the center of Europe, Frankfurt and Munich, it is quite unlikely that our results are due to a local increased prevalence of 16519T. Our results are also not likely due to systematic errors in our procedures, as most of our subjects' genotypes were confirmed by both cyclosequencing and RFLP. Furthermore, in our own control group, 31% have 16519T, although the total is limited to 36 haplogroup H subjects. The polymorphic frequency for 3010A is also highly similar among the two larger control groups, 33% in Americans and 32% in mixed Brits/Americans (Table 1). The proportion does vary in our smaller groups, 22% in Italians and 45% in Finns, but these are non-significant differences (P=0.18) likely due to the small numbers of subjects in those groups (Table 1). Furthermore, since 3010A is present in only 1/63 controls from diverse locations with 16519T, it is highly unlikely that our findings of 3010A in 6/21 CVS and 15/58 MoA subjects with 16519T is an artifact of local polymorphic differences.

We conclude that the common mtDNA polymorphisms 16519T and 3010A are strongly associated with migraine and its variant cyclic vomiting syndrome among individuals with the common West Eurasian haplogroup H, and likely are disease-predisposing. Although only haplogroup H was studied, 16519T and 3010A are found in individuals with a multitude of haplogroups and within all major races. In fact, among Americans of European origin, 16519T is actually slightly more common among non-haplogroup H individuals (34). The effect of these polymorphisms may or may not be dependent upon the background haplogroup, and further study is needed. Our data strengthen the hypothesis that there is a component of mitochondrial dysfunction in migraine and provide additional rationale towards the use of mitochondrial-directed therapies in this condition.

1.6 References
1. Lipton R B, Stewart W F, Von Korff M. The burden of migraine. A review of cost to society. Pharmacoeconomics 1994; 6:215-21.
2. Wessman M, Terwindt G M, Kaunisto M A, Palotie A, Ophoff R A. Migraine: a complex genetic disorder. Lancet Neurol 2007; 6:521-32.
3. Blau J N. Migraine: theories of pathogenesis. Lancet 1992; 339:1202-7.
4. Estevez M, Gardner K L. Update on the genetics of migraine. Hum Genet 2004; 114:225-35.
5. Montagna P, Sacquegna T, Martinelli P, Cortelli P, Bresolin N, Moggio M, et al. Mitochondrial abnormalities in migraine. Preliminary findings. Headache 1988; 28:477-80.
6. Okada H, Araga S, Takeshima T, Nakashima K. Plasma lactic acid and pyruvic acid levels in migraine and tension-type headache. Headache 1998; 38:39-42.
7. Sangiorgi S, Mochi M, Riva R, Cortelli P, Monari L, Pierangeli G, et al. Abnormal platelet mitochondrial function in patients affected by migraine with and without aura. Cephalalgia 1994; 14:21-3.
8. Schoenen J, Jacquy J, Lenaerts M. Effectiveness of high-dose riboflavin in migraine prophylaxis. A randomized controlled trial. Neurology 1998; 50:466-70.
9. Sandor P S, Di Clemente L, Coppola G, Saenger U, Fumal A, Magis D, et al. Efficacy of coenzyme Q10 in migraine prophylaxis: a randomized controlled trial. Neurology 2005; 64:413-5.
10. Sparaco M, Feleppa M, Lipton R B, Rapoport A M, Bigal M E. Mitochondrial dysfunction and migraine: evidence and hypotheses. Cephalalgia 2006; 26:361-72.
11. Couch J R, Bearss C, Verhulst S. Importance of maternal heredity in the etiology of migraine. Neurology 1986; 36 Suppl 1:99.
12. Mortimer M J, Kay J, Jaron A, Good P A. Does a history of maternal migraine or depression predispose children to headache and stomach-ache? Headache 1992; 32:353-5.
13. Ciafaloni E, Ricci E, Shanske S, Moraes C T, Silvestri G, Hirano M, et al. MELAS: clinical features, biochemistry, and molecular genetics. Ann Neurol 1992; 31:391-8.
14. Wang Q, Ito M, Adams K, Li B U, Klopstock T, Maslim A, et al. Mitochondrial DNA control region sequence variation in migraine headache and cyclic vomiting syndrome. Am J Med Genet 2004; 131:50-8.
15. Majamaa K, Finnila S, Turkka J, Hassinen I E. Mitochondrial DNA haplogroup U as a risk factor for occipital stroke in migraine. Lancet 1998; 352:455-6.
16. Li B. Cyclic vomiting: The pattern and syndrome paradigm. J Pediatr Gastroenterol Nutr 1995; 21 Suppl 21:6-10.
17. Boles R G, Adams K, Li B U. Maternal inheritance in cyclic vomiting syndrome. Am J Med Genet 2005; 133:71-7.
18. Fleisher D R. The cyclic vomiting syndrome described. J Pediatr Gastroenterol Nutr 1995; 21 Suppl 21:1-5.
19. Cullen K, Macdonald W B. The periodic syndrome: Its nature and prevalence. Med J Aust 1963; 2:167-172.
20. Abu-Arafeh I, Russell G. Cyclical vomiting syndrome in children: A population based study. J Pediatr Gastroenterol Nutr 1995; 21:454-458.
21. Symon D N, Russell G. The relationship between cyclic vomiting syndrome and abdominal migraine. J Pediatr Gastroenterol Nutr 1995; 21:S42-S43.
22. Li B U, Murray R D, Heitlinger L A, Robbins J L, Hayes J R. Is cyclic vomiting syndrome related to migraine? J Pediatr 1999; 134: 567-572.
23. Boles R G, Williams J C. Mitochondrial disease and cyclic vomiting syndrome. Dig Dis Sci 1999; 44:103 S-107S.
24. Boles R G, Adams K, Ito M, Li BU. Maternal inheritance in cyclic vomiting syndrome with neuromuscular disease. Am J Med Genet 2003; 120:474-82.
25. Boles R G, Powers A L, Adams K. Cyclic vomiting syndrome plus. J Child Neurol 2006; 21:182-8.
26. Ito M, Le S T, Chaudhari D, Higashimoto T, Maslim A, Boles R G. Screening for mitochondrial DNA heteroplasmy in children at risk for mitochondrial disease. Mitochondrion 2001; 1: 269-278.
27. Coelho-Miranda L, Playan A, Artuch R, Vilaseca M A, Colomer J, Briones P, et al. Mitochondrial encephalomyelitis, lactic acidosis and cerebrovascular accidents (MELAS) in pediatric age with the A3243G mutation in the tRNALeu(UUR) gene of mitochondrial DNA. Rev Neurol 2000; 31:804-11.
28. Pronicki M, Sykut-Cegielska J, Mierzewska H, Tonska K, Karczmarewicz E, Iwanicka K, et al. Diversity of clinical symptoms in A3243G mitochondrial DNA mutation (MELAS syndrome mutation). Med Sci Monit 2002; 8:767-73.
29. Boles R G, Chun N, Senadheera D, Wong L-J C. Cyclic vomiting syndrome and mitochondrial DNA mutations. Lancet 1997; 350: 1299-1300.
30. Boles R G, Baldwin E E, Prezant T R. Combined cyclic vomiting and Keams-Sayre syndromes. PediatrNeurol 2007; 36:135-6.
31. The International Classification of Headache Disorders: 2nd edition. Cephalagia 2004; 24 Suppl 1:9-160.
32. Kallela M, Wessman M, Farkkila M. Validation of a migraine-specific questionnaire for use in family studies. Eur J Neurol 2001; 8:61-6.
33. Hermstadt C, Elson J L, Fahy E, Preston G, Turnbull D M, Anderson C, et al. Reduced-median-network analysis of complete mitochondrial DNA coding-region sequences for the major African, Asian, and European haplogroups. Am J Hum Genet 2002; 70:1152-71.
34. Coble M D, Just R S, O'Callaghan J E, Letmanyi I H, Peterson C T, Irwin J A, Parsons T J. Single nucleotide polymorphisms over the entire mtDNA genome that increase the power of forensic testing in Caucasians. Int J Legal Med 2004; 118:137-46.
35. Mishmar D, Ruiz-Pesini E, Golik P, Macaulay V, Clark A G, Hosseini S, et al. Natural selection shaped regional mtDNA variation in humans. Proc Natl Acad Sci USA 2003; 100:171-6.
36. Achilli A, Rengo C, Magri C, Battaglia V, Olivieri A, Scozzari R, et al. The molecular dissection of mtDNA haplogroup H confirms that the Franco-Cantabrian glacial refuge was a major source for the European gene pool. Am J Hum Genet 2004; 75:910-8.
37. Finnila S, Lehtonen M S, Majamaa K. Phylogenetic network for European mtDNA. Am J Hum Genet 2001; 68:1475-84.
38. Ruiz-Pesini, E, Lott, M T, Procaccio, V, Poole J., Brandon M C, Mishmar, D, et al. An enhanced MITOMAP with a global mtDNA mutational phylogeny. Nucleic Acids Research 2007; 35:D823-D828.
39. Shoffner J M, Wallace D C. Oxidative phosphorylation diseases and mitochondrial DNA mutations: diagnosis and treatment. Annu Rev Nutr 1994; 14:535-68.
40. Wallace D C, Brown M D, Lott M T. Mitochondrial DNA variation in human evolution and disease. Gene 1999; 238:211-30.
41. Burnett B B, Gardner A, Boles R G. Mitochondrial inheritance in depression, dysmotility and migraine? J Affect Disord 2005; 88:109-16.
42. Li B U, Lefevre F, Chelimsky G G, Boles R G, Nelson S P, Lewis D W, et al. NASPGHAN Consensus Statement on the Diagnosis and Management of CVS. J Pediatr Gastroenterol Nutr 2007; In Press.
43. Russell M B, Iselius L, Olesen J. Migraine without aura and migraine with aura are inherited disorders. Cephalalgia 1996; 16:305-309.
44. Allard M W, Miller K, Wilson M, Monson K, Budowle B. Characterization of the Caucasian haplogroups present in the SWGDAM forensic mtDNA dataset for 1771 human control region sequences. Scientific Working Group on DNA Analysis Methods. J Forensic Sci 2002; 47:1215-23.
45. Allard M W, Wilson M R, Monson K L, Budowle B. Control region sequences for East Asian individuals in the Scientific Working Group on DNA Analysis Methods forensic mtDNA data set. Leg Med 2004; 6:11-24.
46. Allard M W, Polanskey D, Miller K, Wilson M R, Monson K L, Budowle B. Characterization of human control region sequences of the African American SWGDAM forensic mtDNA data set. Forensic Sci Int 2005; 148:169-79.
47. Allard M W, Polanskey D, Wilson M R, Monson K L, Budowle B. Evaluation of variation in control region sequences for Hispanic individuals in the SWGDAM mtDNA data set. J Forensic Sci 2006; 51:566-73.
48. Navaglia F, Basso D, Fogar P, Sperti C, Greco E, Zambon C F, et al. Mitochondrial DNA D-loop in pancreatic cancer: somatic mutations are epiphenomena while the germline 16519 T variant worsens metabolism and outcome. Am J Clin Pathol 2006; 126:593-601.
49. Murakami H, Ota A, Simojo H, Okada M, Ajisaka R, Kuno S. Polymorphisms in control region of mtDNA relates to individual differences in endurance capacity or trainability. Japan J Physiol 2002; 52:247-256.
50. Rose G, Passarino G, Carrieri G, Altomare K, Greco V, Bertolini S, et al. Paradoxes in longevity: sequence analysis of mtDNA haplogroup J in centenarians. Eur J Hum Genet 2001; 9:701-7.

### Example 2

### Mitochondrial DNA Polymorphisms and Predisposition Towards Depression and Its Co-Morbidities

2.1 Abstract

Data are presented to demonstrate an association between specific mtDNA sequence variants (termed SNPs, pronounced “snips” or single nucleotide polymorphisms) and functional disorders, as well as with functional symptoms in patients with major depressive disorder (MDD) and chronic fatigue syndrome (CFS). We found that the 16519T mtDNA SNP is found statistically more frequently in patients with CFS than in individuals found in the general population. Previously (Example 1), we demonstrated that 16519T, and 3010A on a 16519T background, are found statistically more frequently in patients with migraine and cyclic vomiting syndrome (CVS). Thus, together the data suggest that these mtDNA SNPs increase the chance that an individual will develop these functional conditions.

Furthermore, these SNPs significantly modify the chances that patients diagnosed with functional disorders will report a wide range of specific functional symptoms. Among patients with CFS, 3010A statistically increases the chances of reporting headache, muscle pain, muscle weakness, tingling or numbness, fainting or dizziness, and sleep disturbances. Among patients with MDD, 3010A appears to increase the chance of developing migraine. However, in MDD the opposite SNP, 3010G, quadruples the chance that the individual will suffer from a bowel disorder, and appears to increase the risk several fold for learning disabilities as well.

2.2 Methods

The presence of 16519T versus 16519C and of 3010A versus 3010G was determined by RFLP or cyclosequencing in the following groups of subjects:

1) DNA samples from 295 American MDD subjects from the GenRED (Genetics of Recurrent Early-onset Depression) study were kindly supplied by Dr. Douglas Levinson (Stanford University) and Dr. James Knowles (University of Southern California). The GenRED study comprises 1100 European-American patients with MDD of onset before age 31, all of whom have a first-degree relative with MDD or bipolar 2 of onset before age 41. Among the MDD samples, 112 were of haplogroup H and were further tested. Clinical data on co-morbid functional symptomatology was correlated with the 16519 and 3010 genotypes (Table 5).

2) DNA samples from 162 individuals that meet the 1994 Centers of Disease Control diagnostic criteria for CFS were kindly supplied by Dr. Jonathan Kerr at St. George's University of London. Among the CFS samples, 58 were of haplogroup H and were further tested. Clinical data was available from between 46 to 49 of the subjects (data on all clinical manifestations were not available on all subjects), almost all from London, and was correlated with the 16519 and 3010 genotypes (Table 6).

3) DNA samples from 20 subjects with adult-onset cyclic vomiting syndrome (CVS) were ascertained from the clinic of Dr. R. McCallum, Kansas University in Kansas City. Among the CVS samples, 8 were of haplogroup H and were further tested. The data was combined with that from the 5 adult-onset haplogroup H CVS subjects ascertained from throughout North America from the database of the Cyclic Vomiting Syndrome Association (CVSA). Data was compared against data obtained from the remainder of the CVSA sample (pediatric-onset CVS) and with controls ascertained from the United States, United Kingdom, Italy and Finland.

2.3 Results

2.3.1 Among Patients with MDD, 3010A and 3010G are Associated with Different Co-Morbid Functional Symptoms.

At the present time, 27% of the full data set was analyzed and there is a trend with 3010A being associated with migraine among MDD patients (Table 5), as expected from our previous data. In contrast, the opposite nucleotide of the same SNP, 3010G, is associated with GI disorders among the MDD patients (Table 5). The 3010G+16519C genotype is present in 69% of MDD subjects with and 35% of subjects without any GI disorder (odds ratio=3.92, 95% C.I.=1.3-11.7, P=5×10⁻⁴). A trend was noted suggesting that learning disabilities may also be associated with 3010G (Table 5).

TABLE 5
Pilot GenRED-Derived Data Comparing mtDNA SNPs to Selected Phenotypes

| | | | Any | | Learning | |
|---|---|---|---|---|---|---|
| Genotype | Migraine | P-value | GI Disorder* | P-value | Disability | P-value |
| 3010A | 13/31 | P = 0.18 | 6/31 | P = 0.012 | 1/31 | P = 0.098 |
| | 41.9% | | 19.4% | | 3.2% | |
| 3010G | 23/80 | | 36/80 | | 11/80 | |
| | 28.8% | | 45% | | 13.8% | |
| 16519T | 9/28 | P = 0.97 | 7/28 | P = 0.11 | 2/28 | P = 0.37 |
| | 32.1% | | 25% | | 7.1% | |
| 16519C | 27/83 | | 35/83 | | 10/83 | |
| | 32.5% | | 42.2% | | 12.5% | |

*Colitis, enteritis, or other gastrointestinal disorders

2.3.2 The 16519T mtDNA SNP is Associated with CFS, while Among Patients with CFS, 3010a is Associated with Several Co-Morbid Functional Symptoms.

A pilot study in English subjects found 16519T in 22 of 58 (38%) individuals with chronic fatigue syndrome (CFS) versus in 27% of controls, yielding an odds ratio of 2.0 (95% C.I=1.1−3.7). DNA control samples from London blood donors revealed a proportion of 16519T of 26%, which is essentially the same as the 27% figure obtained from our multi-national (USA, UK, Italy and Finland) control group.

In contrast, 3010A is highly associated with an increase in co-morbid functional symptoms among the subjects with CFS (Table 6).

TABLE 6
3010A is highly associated with a wide variety of functional symptoms
among patients with CFS.
“[0]” = the absence of symptoms, while increasingly higher
levels of somatic symptomatology are represented by higher integer
numbers. “[1+]” = any score 1 or higher. χ2 = chi square
Data on polymorphisms at 3010:

Headache 3010A: data of [0] = 7/21 = 33%, data of [1+] = 14/21 = 67%
Headache 3010G: [0] = 17/25 = 68%, [1+] = 8/25 = 32%
χ2 P = 0.019
Exertional Malaise 3010A: [0] = 6/21 = 29%, [1+] = 15/21 = 71%
Exertional Malaise 3010G: [0] = 14/28 = 50%, [1+] = 14/28 = 50%
χ2 = 0.13; Student's T: A 4.5 + −2.4, G 3.5 + −2.2, P = 0.12 (NS)
Faint/Dizzy 3010A: [0] = 10/21 = 48%, [1+] = 11/21 = 52%
Faint/Dizzy 3010G: [0] = 23/28 = 82%, [1+] = 5/28 = 18%
χ2 P = 0.011
Muscle Weakness 3010A: [0-1] = 7/21 = 33%, [2+] = 14/21 = 67%
Muscle Weakness 3010G: [0-1] = 18/28 = 64%, [2+] = 10/28 = 36%
χ2 P = 0.032
Muscle Weakness A: [0] = 4/21 = 19%, [4+] = 10/21 = 48%
Muscle Weakness G: [0] = 11/28 = 39%, [4+] = 2/28 = 7.1%
χ2 P = 0.0034
Sore Throat 3010A: [0] = 13/21, [1+] = 8/21, [2+] = 3/21
Sore Throat 3010G: [0] = 11/24, [1+] = 13/24, [2+] = 5/24
χ2 P = NS
Cognitive Deficit 3010A: [0] = 5/21, [1+] = 16/21, [2+] = 13/21,
[4+] = 5/21
Cognitive Deficit 3010G: [0] = 9/28, [1+] = 19/28, [2+] = 15/28,
[4+] = 5/28
χ2 P = NS
Muscle Pain 3010A: [0] = 2/21 = 9.5%, [3+] = 13/21 = 62%
Muscle Pain 3010G: [0] = 12/28 = 43%, [3+] = 5/28 = 18%
χ2 P = 0.0011
Joint Pain 3010A: 0 = 9/21, [2+] = 8/21
Joint Pain 3010G: 0 = 14/24, [2+] = 5/24
χ2 P = NS
Sleep Problems 3010A: 0 = 1/22 = 5%, 1-3 = 2/22, [4+] = 19/22 = 86%
Sleep Problems 3010G: 0 = 4/27 = 15%, 1-3 = 10/27, [4+] = 13/27 = 48%
3010A: [0-3] = 3/22 = 14%
3010G: [0-3] = 14/27 = 45%
χ2 ([0-3] vs. [4+]) P = 0.0052
GI Problems 3010A: [0] = 7/21 = 33%, [1] = 8/21, [2+] = 6/21;
[1+] = 67%
GI Problems 3010G: [0] = 16/28 = 57%, [1] = 6/28, [2+] = 6/28;
[1+] = 43%
χ2 ([0] vs. [1+]) P = 0.10 (NS)
Numbness/Tingling 3010A: [0] = 9/21 = 43%, [2+] = 7/21 = 33%
Numbness/Tingling 3010G: [0] = 18/24 = 75%, [2+] = 2/24 = 8.3%
Fisher exact P = 0.026

The chronic fatigue data is very encouraging, and provides substantial support to our data demonstrating that 3010A is associated with a higher degree of somatic/functional symptomatology across the board, with the possible exception of bowel symptoms.

2.3.3 Data From Adult-Onset Versus Pediatric-Onset CVS Patients.

Analysis of data from adult-onset (18 years+) versus pediatric-onset of vomiting episodes in North American subjects with cyclic vomiting syndrome is presented below.

TABLE 7
Adult versus pediatric onset in CVS, pilot statistics
The 3010G + 16519C genotype was found in:

| | | |
|---|---|---|
| Adult-onset CVS | 10/13 | 77% |
| Child-onset CVS | 2/17 | 11% |
| Controls | 95/231 | 41% |

| | |
|---|---|
| P adult-onset CVS vs. child-onset CVS | P = 3.1 × 10⁻⁴ |
| P adult-onset CVS vs. controls | P = 0.011 |
| P child-onset CVS vs. controls | P = 0.017 |

2.3.4 Data From SNPs other than 16519 and 3010.

Although 16519 and 3010 comprise a substantial proportion of the inherited risk to develop functional disease and functional symptoms, they do not comprise all such risk. The data demonstrate that additional functional-disease associated mtDNA SNPs likely exist, some of which have been identified. These other SNPs were found together in sets, in both patients with functional disorders and in controls (Tables 3 and 8), which does not allow at present the determination of which one, or ones, in said sets are pathogenic and which one, or ones, are neutral markers. The 2259T, 4745G, 7337A, 13326C, 13680T, 14831A and 14872T cluster was found in CVS subject numbers 2, 4 and 10, and the 239C, 4727G and 9380A cluster was found in CVS subject numbers 13 and 17 (Table 3). Among 16519T haplogroup H subjects, further data analysis revealed that the 13326C SNP was found in 4/30 CVS and in 3/22 CFS patients versus in 1/52 controls (P=0.03). The sample size of our pilot data is underpowered such that few of the other SNPs in the clusters achieved statistical significance, but a trend suggesting that these clusters are associated with CVS is apparent in the data shown in Table 3.

2.4 Discussion

This analysis revealed that mtDNA SNPs 239C, 2259T, 3010A, 3010G, 4727G, 4745G, 7337A, 9380A, 13326C, 13680T, 14831A, 14872T, 16519T, and 16519C are associated with functional disease, especially the SNPs at 3010 and 16519. The data further showed that 3010 is associated with co-morbid functional symptoms among subjects with a diagnosis of a functional disorder. Identifying mtDNA sequence variants that predispose towards depression and co-morbid functional symptomatology will help identify subgroups of patients where mitochondrial dysfunction is a particularly important pathogenic mechanism. These individuals may respond differently to medications, and may benefit from mitochondrial-targeted treatments, including frequent feedings, co-enzyme Q10, riboflavin and antioxidants. Our data and clinical experience suggest that most patients with functional symptoms do indeed respond favorably to these treatments.

These studies identify 16519T at an increased prevalence versus controls in functional disorders like pediatric-onset CVS in North Americans ascertained from the CVSA, migraine headache in Germans seen in headache clinics, and chronic fatigue syndrome in English patients.

3010A was identified at an increased prevalence versus controls in subjects with 16519T in functional disorders like pediatric-onset CVS in North Americans ascertained from the CVSA and in migraine headache in Germans seen in headache clinics. 3010A was identified at an increased prevalence in English patients with chronic fatigue syndrome among the subset of patients reporting the following co-morbid functional symptoms: headache, muscle pain, muscle weakness, numbness or tingling, fainting or dizziness and/or sleep problems. A trend was found for 3010A being more common in American adults with recurrent early-onset major depressive disorder among those reporting any kind of headache.

3010G was identified at an increased prevalence in patients with the following gut dysmotility-related phenomenon: in American adults with recurrent early-onset major depressive disorder among those reporting any gastrointestinal disorder versus among those reporting no GI disorders, and in adult-onset CVS in North Americans versus in controls. A trend was found for 3010G being more common in American adults with recurrent early-onset major depressive disorder among those reporting learning disabilities.

These studies show that 16519T predisposes towards the development of multiple functional disorders; that on a 16519T background, 3010A predisposes towards the development of multiple functional disorders; that among patients diagnosed with functional conditions, 3010A predisposes towards multiple manifestations of functional symptomatology; and that 3010G predisposes individuals towards gastrointestinal-related functional symptomatology.

The results and conclusions are further summarized in Table 8 below.

TABLE 8
Summary of Patient Groups, Polymorphisms, and Analyses when finding a
Polymorphism in a Patient Group as indicated.

| Patient Group: | Polymorphism: | Analysis: |
|---|---|---|
| Haplogroup H | 16519T | 6-fold more likely to have CVS |
| children | | |
| Haplogroup H adults | 16519T | 2 and 4-fold more likely to have |
| | | CFS or migraine, respectively |
| Haplogroup H | 16519T + 3010A | 17-fold more likely to have CVS |
| children | | |
| Haplogroup H adults | 16519T + 3010A | 15-fold more likely to have |
| | | migraine |
| Haplogroup H adults | 16519C + 3010G | A few fold more likely to have CVS |
| Haplogroup H adults | 3010A | 3 to 7-fold more likely to have |
| diagnosed with | | headache, fainting or dizziness, |
| chronic fatigue | | muscle pain, muscle weakness, |
| syndrome | | numbness or tingling, or sleep |
| | | problems |
| Haplogroup H adults | 3010G | 4-fold more likely to have any |
| diagnosed with | | bowel disorder; possibly more likely |
| depression | | to have learning disabilities |
| Haplogroup H adults | 3010A | Possibly more likely to have |
| diagnosed with | | headaches |
| depression | | |
| Haplogroup H | Any of the group: | More likely to have child-onset |
| individuals | 2259T, 4745G, 7337A, | CVS, CFS or migraine |
| | 13326C, 13680T, | |
| | 14831A or 14872T | |
| Haplogroup H | Any of the group: | More likely to have child-onset |
| individuals | 239C, 4727G or 9380A | CVS, CFS or migraine |

2.5 References
1. Boles R G, Adams K, Li B U. K (2005): Maternal inheritance in cyclic vomiting syndrome. Am J Med Genet 133A:71-7.
2. Boles R G, Burnett B B, Gleditsch K, Wong S, Guedalia A, Kaariainen A, Eloed J, Stern A and Brumm V (2005): A high predisposition to depression and anxiety in mothers and other matrilineal relatives of children with presumed maternally inherited mitochondrial disorders. Am J Med Genet B Neuropsychiatr Genet 137B:20-4.
3. Bumet B B, Gardner A, Boles R G (2005): Mitochondrial inheritance in depression, dysmotility and migraine? J Affect Disord 88:109-16.
4. Gardner A, Boles R G (2005): Is a “Mitochondrial Psychiatry” in the future? A review. Current Psychiatry Reviews 1:255-71.
5. Gardner A, Boles R G (2008): Mitochondrial energy depletion in depression with somatization. Psychother Psychosom 77:127-9.
6. Gardner A, Boles R G (2008): Symptoms of somatization as a rapid screening tool for mitochondrial dysfunction in depression. Biopsychosoc Med 2:7 [Epub ahead of print].
7. Holmans P, Weissman M M, Zubenko G S, Scheftner W A, Crowe R R, Depaulo J R Jr, Knowles J A, Zubenko W N, Murphy-Eberenz K, Marta D H, Boutelle S, McInnis M G, Adams P, Gladis M, Steele J, Miller E B, Potash J B, Mackinnon D F, Levinson D F (2007): Genetics of recurrent early-onset major depression (GenRED): final genome scan report. Am J Psychiatry 164:248-58.
8. Kato T (2007). Molecular genetics of bipolar disorder and depression. Psychiatry Clin Neurosci. 61:3-19.
9. Levinson D F, Evgrafov O V, Knowles J A, Potash J B, Weissman M M, Scheftner W A, Depaulo J R Jr, Crowe R R, Murphy-Eberenz K, Marta D H, McInnis M G, Adams P, Gladis M, Miller E B, Thomas J, Holmans P (2007): Genetics of recurrent early-onset major depression (GenRED): significant linkage on chromosome 15q25-q26 after fine mapping with single nucleotide polymorphism markers. Am J Psychiatry 164:259-64.
10. Schur E A, Afari N, Furberg H, Olarte M, Goldberg J, Sullivan P F, Buchwald D (2007): Feeling bad in more ways than one: comorbidity patterns of medically unexplained and psychiatric conditions. J Gen Intern Med 22:818-21.

### Example 3

### Association of Three Common Mitochondrial DNA Polymorphisms with Functional Gastrointestinal Disorders and Gastrointestinal Motor and Sensory Functions

3.1 Abstract

Background: Familial aggregation of irritable bowel syndrome (IBS) frequently involves mothers and their children. Since mitochondrial DNA (mtDNA) is exclusively maternally inherited, mtDNA polymorphisms could confer risk to the development of IBS. The mtDNA single nucleotide polymorphisms (SNPs) 16519C>T and 3010G>A are associated with migraine and cyclic vomiting syndrome. Hypothesis: mtDNA 16519C>T and 3010G>A SNPs are associated with functional gastrointestinal disorders (FGIDs) and with gastrointestinal sensorimotor functions. Methods: The mitochondrial genome was first tested for the 7028C mtDNA polymorphism (defining haplogroup H) in 699 participants (patients or controls), and those with 7028C were subsequently genotyped at 16519 and 3010. The phenotype of all participants was based on symptom phenotype (validated questionnaires using consensus criteria) and gastrointestinal physiology using validated motor and sensory studies. Results: Constipation, and possibly mixed, IBS are less prevalent in individuals with the 7028C mtDNA polymorphism than in individuals with 7028T. Conversely, 7028C is associated with higher maximum tolerated volume (satiation) compared to 7028T. Among those with 7028C, non-specific abdominal pain (chronic abdominal pain or dyspepsia) was significantly associated with 3010A compared to 3010G (odds ratio 3.3, p=0.02), and slower gastric emptying was statistically associated with 3010A. There were no significant associations of genotype with stomach volumes, small bowel or colonic transit, rectal compliance or abnormal motor or sensory functions. Conclusion: These data suggest that genetic variation in mtDNA may be associated with satiation volume, gastric emptying and/or pain thresholds; further studies of mtDNA in appetite regulation and larger numbers of patients with functional GI conditions are warranted.

3.2 Introduction

There is evidence of familial aggregation of irritable bowel syndrome (IBS), typically involving mothers and their children (1,2). In a recent study of familial aggregation (2), there was a significant odds ratio for mothers and sisters of IBS probands to be affected with IBS (odds ratios 3.4 and 3.1, respectively). Studies exploring the genetic epidemiology of functional gastrointestinal disorders (FGID) have generally addressed the potential association with single mechanisms such as receptors, transporters and translation or transduction mechanisms. To date, there is no definite evidence that any single genetic defect is associated with functional gastrointestinal disorders. For example, in the case of the most frequently reported association of genetic variation in IBS, a meta-analyses of several studies on 5-HTTLPR that included patient groups of Asian or European ethnicity did not reveal a significant association of the genetic variation with IBS relative to health in people from the same ethnic groups (3). On the other hand, there is increasing evidence of an association between GNβ3 polymorphisms and functional dyspepsia (4-7). Given the familial aggregation data and the evidence suggesting an association of genetic variation with functional dyspepsia, further studies of the association of common genes with FGID are warranted.

The nerve, muscle and inflammatory cells that may be involved in the mechanisms underlying the development of functional gastrointestinal disorders (8) have high-energy requirements and are thus cell types that are frequently affected in the mitochondrial disorders. Mitochondria are cytoplasmic organelles that produce the bulk of the ATP for cellular energy needs. Mitochondrial proteins are encoded on both the nuclear DNA (chromosomes) as well as the 16-kilobase mitochondrial DNA (mtDNA). Thus, sequence variants (polymorphisms) that adversely affect energy metabolism and predispose towards disease pathogenesis theoretically could be on either or both of those genomes (9,10). The cytoplasmic-located mtDNA generally is derived solely from the ova without recombination, and individuals related through women carry an identical mtDNA sequence in the absence of a recent mutation. A disease like IBS that has a significant odds ratio to aggregate in mothers and sisters (2), suggesting bias toward maternal transmission, may conceivably result from predisposing mtDNA sequence variants.

The functional disorder, cyclic vomiting syndrome (CVS), generally demonstrates maternal inheritance (11), and mtDNA sequence variants in that condition have been reported (12-15). Conversely, patients with mtDNA disorders frequently suffer from symptoms that overlap with functional gastrointestinal conditions (16,17).

In a previous study by Boles et al., two common mtDNA SNPs, 16519C>T and 3010G>A, were found to be associated with CVS and migraine among patients with the mtDNA single nucleotide polymorphism (SNP) 7028C [haplogroup H (11)]. Given the important role of mitochondria in neuromuscular function, inflammation and the possible role of these mechanisms in FGID (8), we explored the hypothesis that these mtDNA SNPs are associated with IBS, functional constipation, functional diarrhea, chronic abdominal pain and functional dyspepsia. To explore this hypothesis, we sought the presence of associations between the three common mtDNA SNPs and symptom phenotype of FGIDs and gastrointestinal motor and sensory functions.

3.3 Materials and Methods

3.3.1 Overall Design

We assessed symptom phenotype using consensus criteria with validated questionnaires that assessed gastrointestinal symptoms (18) in all participants (which included an assessment of somatic symptoms in a majority [63%] of the participants), and gastrointestinal function using validated motor and sensory studies: satiation nutrient drink test to estimate maximum tolerated volume (MTV, n=116), gastrointestinal (n=268) and colonic (n=172) transit of solid food and residue by dual isotope scintigraphy, gastric volume (fasting and post-meal accommodation) by ^{99m}Tc-SPECT (n=228), and rectal compliance and sensation by barostat (n=116).

3.3.2 Participants

This study assessed 463 patients with FGIDs [Rome II positive; 19 chronic abdominal pain, 175 diarrhea-predominant IBS (IBS-D) or functional diarrhea, 155 constipation-predominant IBS (IBS-C) or functional constipation, 84 IBS-mixed, and 33 dyspepsia] and 233 healthy volunteers recruited to studies of symptom phenotype and genotype from 2000-2007 (5,19-21). All participants were residents of the region within 150 miles of Rochester, Minn. Participants had been recruited for the original studies (5,19-21) by means of letters or public advertisements and had signed informed consent for the respective studies.

The inclusion criteria and characteristics of each patient group appear in the original studies; all patients fulfilled Rome II criteria. For example, the group with chronic (functional) abdominal pain had abdominal pain of at least 12 weeks duration (not necessarily consecutive) in the absence of bowel dysfunction in order to differentiate from IBS. In addition, functional dyspepsia patients were identified by upper abdominal pain and discomfort related to food ingestion (20). Use of the database from which this analysis was conducted was reviewed and approved by the Mayo Clinic Institutional Review Board, and all participants had given permission for research studies based on medical records and their DNA samples.

The validated bowel symptom questionnaire, review of the electronic medical record (SM), or direct physician interview and examination (MC) were used to characterize the subtype of FGID. The physiological measurements have been used extensively to characterize motor and sensory functions in patients with FGID and to document the effects of pharmacological agents on these functions in health and disease states.

3.3.3 Satiation by the Nutrient Drink Test

A standardized Ensured® (1 Kcal/mL, 11% fat, 73% carbohydrate and 16% protein) drink test (22) was used to measure satiation and postprandial symptoms of nausea, bloating, and pain 30 minutes after the meal in 116 participants.

3.3.4 Gastrointestinal and Colonic Transit by Scintigraphy

An adaptation of our established combined scintigraphic method was used and provided measurements of gastric (n=268), small bowel (n=219) and colonic transit (n=172). In 98 participants, gastric emptying was measured by the scintigraphic method using the same radiolabeled meal and scans were obtained over the first 4 hours.

The data were generally acquired in single center pharmacodynamic studies, the genetic association studies detailed above (19-21) or pharmacogenetics studies of the pharmacodynamic response to the drugs, alosetron (23) or clonidine (24). Briefly, ¹¹¹In adsorbed on activated charcoal particles was delivered to the colon by means of a methacrylate-coated, delayed-release capsule administered by mouth. The capsule was ingested following an overnight fast. After the capsule emptied from the stomach (documented by its position relative to radioisotopic markers placed on the anterior iliac crests), a radiolabeled meal was ingested. In this meal, ^{99m}Tc sulfur colloid was used to label two scrambled eggs that were eaten with one slice of whole wheat bread and one glass of whole milk (total 300 kcal). This meal facilitated measurement of gastric and small bowel transit. Subjects ingested standardized meals for lunch and dinner at 4 and 8 hours after the radiolabeled meal. Abdominal scans were obtained every hour for the first 6 hours, and at 8, 24, and 48 hours after ingestion of the ¹¹¹In capsule. The performance characteristics of these tests were summarized elsewhere (25).

3.3.5 Transit Data Analysis

A variable region of interest program was used to quantitate the counts in the stomach and each of four colonic regions: ascending, transverse, descending, and combined sigmoid and rectum. These counts were corrected for isotope decay, tissue attenuation, and downscatter of ¹¹¹In counts in the ^{99m}Tc window.

Gastric emptying t_{1/2} is a measure of the time for 50% of the radiolabeled meal (identifiable by radiolabeled tracer) to empty from the stomach. Overall colonic transit was summarized as the colonic geometric center (GC) at specified times: the GC is the weighted average of counts in the different colonic regions [ascending (AC), transverse (TC), descending (DC), rectosigmoid (RS)] and stool, respectively 1 to 5. Thus, at any time, the proportion of counts in each colonic region is multiplied by its weighting factor as: (% AC×1+% TC×2+% DC×3+% RS×4+% stool×5)/100=geometric center. Thus, a higher GC reflects a faster colonic transit.

The primary transit endpoints were the gastric emptying t_{1/2} and colonic geometric center at 24 hours (GC24).

3.3.6 Gastric Volume by ^{99m}Tc-SPECT

Gastric volume was measured in 228 participants using a method developed and validated in our lab (26). We measured the gastric volume during fasting and post-300 ml Ensure® (300 kcal, Ross Labs, Abbott Park, Ill.). This method uses SPECT after intravenous administration of ^{99m}Tc-sodium pertechnetate (0.12mCi/kg), which is taken up by the gastric mucosa. The camera (SMV-GE, Fairfield, Conn.) rotates around the thorax and abdomen with the participant lying in the supine position. The stomach was identified in the transaxial SPECT images and separated from background using a semi-automated segmentation algorithm. A three-dimensional rendering of the stomach and its volume was obtained using the AVW 3.0 (Biomedical Imaging Resource, Mayo Foundation, Rochester, Minn.) image processing libraries. The primary endpoints were fasting and postprandial gastric volume.

3.3.7 Rectal Compliance and Sensation by Barostat

These studies were conducted in 112 subjects (87 patients with IBS and 25 healthy controls) who presented to the research center after bowel preparation (Fleet® phosphate enema, self-administered at least 1 hour before reporting to the center) and an overnight fast. The studies were conducted as described in detail elsewhere (27). A catheter with a polyethylene bag (MUI Scientific, Mississauga, Ontario, Canada), was inserted into the rectum so that the middle of the balloon was about 10 cm from the anal verge. Subjects were placed in a semi-prone position and the foot end of the bed was elevated 15 degrees. The bag was then unfolded by transiently inflating it with 75 ml of air. The catheter was connected to a barostat (G&J Electronics Inc., Toronto, Ontario, Canada) and the pressure in the bag was increased from 4 mmHg in steps of 1 mmHg for 1 minute per step until respiratory excursions were observed. The baseline operating pressure was defined as 2 mmHg above the minimal distension pressure at which respiratory excursions were clearly recorded from the barostat tracing. An initial “conditioning” distension of the rectum was performed with pressure increased from 0 to 20 mmHg in steps of 4 mmHg for 15 seconds per step. This renders subsequent assessments of compliance and perception more reproducible (28). The bag was then deflated to 0 mmHg and the subjects were allowed to rest for 10 minutes.

Rectal sensory thresholds were measured by ramp inflation, starting at 0 mmHg and increasing in steps of 4 mmHg for 1 minute per step to a maximum of 60 mmHg. Thresholds for first sensation, gas, urgency, and pain were indicated by the subjects pressing a button at the distension pressure at which sensations were perceived. Ramp inflation was terminated when the subjects reported the first sensation of pain. Following this procedure, the bag was deflated to the baseline operating pressure and the subjects were allowed to rest for 10 minutes.

Rectal sensory ratings were measured using phasic distensions of 12, 24, 30 and 36 mmHg above baseline operating pressure applied once in random order. The order was provided by the study statistician (ARZ). Each distention was maintained for 60 seconds with an inter-stimulus interval of 2 minutes, during which time the balloon was deflated to the baseline operating pressure. Subjects were asked to mark separate 100 mm visual analog scales (VAS) 30 seconds after the onset of the distension for the sensations of gas, urgency, and pain. These scales were anchored at the ends by the descriptions ‘unnoticeable’ and ‘unbearable’. Pressure was released if the subject reported greater than 80 mm of pain on the VAS scale, and higher distensions were not subsequently administered. Immediately prior to the sensory testing, the participants filled in 100 mm VAS assessing their current state of “tiredness”, “worry”, “peace”, and “activity”. The methods and analysis have been described in detail elsewhere (29).

The following measurements were derived: (i) the sensory thresholds for first sensation, gas, urgency, and pain during ascending method of limits and (ii) the gas, urgency, and pain scores in response to the four random phasic distensions (12, 24, 30 and 36 mmHg above baseline operating pressure) delivered in randomized order according to a scheme generated by the study statistician and communicated to the technologist on the day of the sensation test.

3.3.8 mtDNA Genotyping Methods

DNA was isolated from blood by standard methods in 699 of the 701 participants. The mtDNA haplogroups denote sets of matrilineal ancestry tens of thousands of years old. The presence or absence of the 7028C polymorphism that defines haplogroup H was determined by PCR/restriction fragment length polymorphism analysis following AluI digestion, with primer sequences (F: TTTCGGTCACCCTGAAGTTTA (SEQ ID NO:5); and R: AGCGAAGGCTTCTCAAATCAT (SEQ ID NO:6)). The West Eurasian haplogroup H is well suited for genetic association studies due to the relative lack of intra-group sequence variability and high prevalence. As per the previous study from the Boles laboratory (11), limiting the present study to subjects with haplogroup H substantially decreases background sequence variability and correspondingly increases statistical power. It is also practical, as haplogroup H is the most common among European-derived populations, including prevalence rates of about 45% in the native population of Germanic countries and in about 33% of North Americans of apparent European ancestry (30).

Participants with haplogroup H were tested for the 16519C>T and 3010G>A polymorphisms by PCR/restriction fragment length polymorphism (16519: HaeIII F: GGATGACCCCCCTCAGATA (SEQ ID NO:1); R: CTTATTTAAGGGGAACGTG (SEQ ID NO:2); 3010: BccI F: CATGCTAAGACTTCACCA (SEQ ID NO:3); R: TCGTTGAACAAACGAACC (SEQ ID NO:4)). Genotypes were confirmed by random direct sequencing at Mayo Clinic's DNA Sequencing Core Facility using Applied Biosystems BigDye® terminator v1.1 cycle sequencing chemistry and analyzed on Applied Biosystems 3730XL DNA Analyzer. 16519T is designated herein as the “polymorphism”, despite 16519C being the reference nucleotide (31), because 16519T is both the ancestral nucleotide as well as the most common nucleotide seen in all major human races.

3.3.9 Statistical Analysis

The statistical analyses were structured in three parts: First, were associations with overall haplogroup status (7028C=haplogroup H vs. 7028T=all non-H haplogroups); second, within the H haplogroup, the associations with 3010G vs. 3010A, and separately, 16519C vs. 16519T, were evaluated. mtDNA differs in many substantial ways from nuclear DNA. For example, the polymorphisms in mtDNA are in complete linkage disequilibrium as mtDNA never recombines. Moreover, the 16519 locus is in the region that controls replication for the entire mtDNA, including 3010. Thus, third we explored associations of symptom phenotype and gastrointestinal functions with the combinations of different genotypes at the 16519 locus and the different genotypes at the 3010 locus, that is GC, GT, AC, and AT, among participants with haplogroup H.

3.3.10 Symptom Phenotype

The overall univariate association of genotype with symptom phenotype was assessed using contingency table analyses (χ² test), combining all FGIDs into one group and, separately, using the individual FGID subtypes. Odds ratios (95% CIs) for each symptom phenotype (compared to healthy controls) and the mtDNA genotypes (e.g., 7028C relative to 7028T, 3010G relative to 3010A, and 16519T relative to 16519C) were estimated using multiple logistic regression, adjusting for gender. Due to the small number of subjects in the AT combination, the multiple logistic regression model predicting individual symptom phenotypes was not examined for the genotype 3010A and 16519T combination.

3.3.11 GI Motor Function

The association with GI motor function was assessed using analysis of covariance (ANCOVA), adjusting for age, gender, and body mass index (BMI). These analyses were done using all subjects, with physiology data to examine these associations separately for gastric emptying, small bowel transit, colonic transit, nutrient drink test challenges (maximum tolerated volume and aggregate symptom score 30 minutes post satiation), and gastric volumes as measured by SPECT.

3.3.12 Rectal Sensation

The associations with each rectal sensation threshold type (gas, urgency and pain) was assessed using the log-rank test for specific pairs (e.g., 7028C vs. T, 3010G vs. A, and 16519T vs. C) and summarized as median (% censored) based on the Kaplan-Meier product limit method. The association between colonic sensation VAS ratings scores and genotype was based on a repeated measures ANCOVA (the repeated factor being the multiple pressure distension levels) and the Wilcoxon rank sum test at specific distension levels, separately for gas, urgency and pain sensation types.

The aim in these hypotheses-generating analyses was to explore potential associations that would warrant further study and thus no adjustment in the alpha level for multiple tests was made. In particular, p-values between 0.05 and 0.1 were considered suggestive of potential associations that might deserve further study with larger numbers of subjects.

Results

Haplogrouping of Study Participants

In this predominantly Caucasian cohort, 42.9% carried the 7028C SNP (haplogroup H). As expected, the 16519 polymorphism was associated with the 3010 polymorphism (p<0.001).

3.4.2 Association of mtDNA Genotype and Symptom Phenotype

Table 9 shows a summary of demographic, somatic symptom scores and physiological data. In Table 10, the proportion of FGID phenotypes in H and non-H haplogroups are reported; within the H haplogroup, data for 300 participants are subdivided further according to 3010 and 16519 genotype.

TABLE 9
Demographic, Somatic, Motor and Satiation Data (mean ± SEM)

| | GROUP: | | | | | |
|---|---|---|---|---|---|---|
| | Non-H | H | 3010 G | 3010 A | 16519 C | 16519 T |
| | Haplogroup | Haplogroup | genotype | genotype | genotype | genotype |
| Variable: | n = 399 | n = 300 | n = 190 | n = 110 | n = 210 | n = 90 |
| BMI, kg/m² | 27.3 ± 0.3 | 27.4 ± 0.3 | 27.4 ± 0.4 | 27.3 ± 0.5 | 27.3 ± 0.4 | 27.5 ± 0.6 |
| Age | 41.4 ± 0.7 | 43.8 ± 0.8 | 42.7 ± 1.0 | 45.6 ± 1.4 | 44.7 ± 1.0 | 41.8 ± 1.4 |
| Somatic | 0.66 ± 0.04 | 0.57 ± 0.04 | 0.55 ± 0.05 | 0.60 ± 0.06 | 0.58 ± 0.05 | 0.53 ± 0.06 |
| symptom | | | | | | |
| score | | | | | | |
| % Stomach | 52.1 ± 1.4 | 52.1 ± 1.8 | 48.1 ± 1.9 | 58.0 ± 3.2 | 54.1 ± 2.2 | 46.9 ± 2.52 |
| emptied @120 min | | | | | | |
| % Colonic | 27.2 ± 2.8 | 22.5 ± 3.3 | 28.8 ± 4.6 | 13.5 ± 4.1 | 18.9 ± 3.8 | 31.0 ± 6.2 |
| filling @ 6 hr | | | | | | |
| Colon GC @ | 2.45 ± 0.1 | 2.80 ± 0.14 | 2.72 ± 0.15 | 2.92 ± 0.28 | 2.81 ± 0.18 | 2.77 ± 0.22 |
| 24 hr | | | | | | |
| Max^{m} | 1021 ± 36 | 1181 ± 50 | 1221 ± 63 | 1096 ± 78 | 1154 ± 68 | 1224 ± 72 |
| tolerated | | | | | | |
| volume, mL | | | | | | |
| Aggregate | 171.9 ± 8.3 | 197.3 ± 14.1. | 201.0 ± 15.4 | 189.2 ± 30.6 | 204.6 ± 18.1 | 185.8 ± 22.9 |
| symptom | | | | | | |
| score | | | | | | |
| Fasting gastric | 232.1 ± 7.1 | 245.5 ± 8.5 | 244.7 ± 11.4 | 246.9 ± 12.6 | 240.2 ± 8.6 | 258.6 ± 20.2 |
| volume, mL | | | | | | |
| Δ PP-fasting | 508.7 ± 8.4 | 506.9 ± 9.1 | 513.4 ± 10.7 | 496.8 ± 16.4 | 505.5 ± 11.7 | 510.3 ± 13.2 |
| gastric vol, | | | | | | |
| mL | | | | | | |

TABLE 10
Proportion (%) of FGID Phenotypes in Haplogroup H and Combined Non-H
Haplogroups (within the H haplogroup, data for 300 participants are subdivided
further according to 3010 and 16519 genotypes; data are mean ± SEM or
proportion (%) of FGID phenotypes within each haplogroup or genotype)

| | | % | % A- | % C- | % D- | % | | % GE | MTV, |
|---|---|---|---|---|---|---|---|---|---|
| Haplogroup | N | FGID | IBS | IBS | IBS | FD | % CAP | @ 120 min | mL |
| All non-H | 399 | 69 | 14 | 25 | 23 | 4 | 3 | 52 ± 1 | 1021 ± 36 |
| haplogroups | | | | | | | | | |
| (7028T) | | | | | | | | | |
| Haplogroup | 300 | 64 | 10 | 19* | 27 | 5 | 3 | 52 ± 2 | 1181 ± 50* |
| H (7028C) | | | | | | | | | |
| 3010G | 190 | 61 | 10 | 21 | 25 | 4 | 1 | 48 ± 2** | 1221 ± 63 |
| genotype | | | | | | | | | |
| 3010A | 110 | 69 | 10 | 15 | 31 | 8 | 5 | 58 ± 3 | 1096 ± 78 |
| genotype | | | | | | | | | |
| 16519C | 210 | 65 | 10 | 19 | 27 | 7 | 2 | 54 ± 2 | 1154 ± 68 |
| genotype | | | | | | | | | |
| 16519T | 90 | 61 | 11 | 18 | 29 | 2 | 1 | 47 ± 3 | 1224 ± 72 |
| genotype | | | | | | | | | |

A = alternating (“mixed”),
C = constipation,
D = diarrhea;
*p < 0.05 vs. all non-H haplogroups;
**p = 0.04 vs. A genotype;
FD = Functional Dyspepsia

A somewhat lower odds for any FGID (compared to healthy controls) in haplogroup H (relative to all other haplogroups) was observed [OR (95% CI)]=0.8(0.6, 1.1) with, specifically, a lower odds for C-IBS [OR=0.6 (0.4, 0.9), p=0.02] and a somewhat lower odds ratio for IBS-alternating (p=0.052), as shown in FIG. 9.

An increased odds for any FGID (compared to controls) was observed (Table 11) in 3010A [relative to G, OR=1.6 (1.0, 2.8), p=0.06], specifically, an increased odds for D-IBS [OR=1.7 (0.9, 3.2), p=0.09]. If one were to consider chronic abdominal pain and dyspepsia as a combined, non-specific abdominal pain group, there was an increased odds for this condition (compared to controls) in the 3010A genotype [relative to the 3010G genotype, OR=3.2 (1.2, 8.0), p=0.02].

TABLE 11
Association of Mitochondrial DNA 16519 and 3010 Genotypes with FGID Overall

| | | | Chronic | Constipation | Diarrhea | | | |
|---|---|---|---|---|---|---|---|---|
| Mitochondrial | Any | | Abdominal | and | and | | | |
| DNA | FGID | IBS-A | Pain | IBS-C | IBS-D | Dyspepsia | Health | Total |
| 16519C | 136 | 20 | 6 | 40 | 56 | 14 | 74 | 210 |
| % of Disease | 71.20 | 66.67 | 85.71 | 71.43 | 68.29 | 87.5 | 67.89 | |
| Group | | | | | | | | |
| 16519T | 55 | 10 | 1 | 16 | 26 | 2 | 35 | 90 |
| % of Disease | 28.80 | 33.33 | 14.29 | 28.57 | 31.71 | 12.5 | 32.11 | |
| Group | | | | | | | | |
| 3010G | 115 | 19 | 2 | 39 | 48 | 7 | 75 | 190 |
| % of Disease | 60.21 | 63.33 | 28.57 | 69.64 | 58.54 | 43.75 | 68.81 | |
| Group | | | | | | | | |
| 3010A | 76 | 11 | 5 | 17 | 34 | 9 | 34 | 110 |
| % of Disease | 39.79 | 36.67 | 71.43 | 30.36 | 41.46 | 56.25 | 31.19 | |
| Group | | | | | | | | |

No significant phenotypic associations were detected for 16519C versus T, for any genotype and somatic symptom scores.

3.4.3 Association of Haplogroup H with Gastrointestinal Functions

There were no significant associations for the presence of haplogroup H (vs. all non-H haplogroups) with any other motor functions (gastrointestinal transit, specifically CF@6 hrs, GC@24 hrs, GC@48 hrs), or rectal compliance, aggregate symptom score after nutrient drink test, fasting volume (SPECT), and delta volume [fasting minus fed (Table 10)]. In contrast, there was a significant association (p=0.037) of haplogroup status with satiation (the maximum tolerated volume of Ensure® ingested during the nutrient drink test, Table 10), with higher volumes observed in the H haplogroup overall. There was also a significant association between gastric emptying at 120 minutes and 3010 (p=0.043), with slower emptying in the 3010A genotype (vs. 3010G).

There were no significant associations detected for rectal compliance (p=0.09), sensation thresholds or sensation ratings in people carrying haplogroup H versus all other non-H haplogroups (Table 12). However, while the overall sensation of gas ratings was not significant (p=0.156), sensation ratings for gas at 24 and 30 mmHg distension were lower in the H vs. non-H haplogroups (p=0.032 and 0.031, respectively). Similarly, an overall association of the four genotypes (within H haplogroup) with the threshold sensation for gas was not significant (p=0.101), and the repeated measures analysis of variance for the sensory rating scores did not detect any significant associations (Table 12).

TABLE 12
Rectal Compliance and Sensation in Different Haplotype and Genotype Groups

| | Group | | | | | |
|---|---|---|---|---|---|---|
| | Non-H | H | 3010 G | 3010 A | 16519 C | 16519 T |
| | haplogroup | haplogroup | genotype | genotype | genotype | genotype |
| Variable | n = 75 | n = 41 | n = 28 | n = 13 | n = 25 | n = 16 |
| Compliance, Pr ½, | 12.6 ± 0.6 | 14.2 ± 0.7 | 14.0 ± 0.9 | 14.4 ± 1.0 | 13.4 ± 0.9 | 15.3 ± 0.9 |
| mmHg | | | | | | |
| Median Gas threshold, | 12 (8%) | 12 (15%) | 12 (7%) | 16 (31%) | 14 (17%) | 12 (12%) |
| mmHg (% censored) | | | | | | |
| Median Urgency | 16 (0%) | 20 (0%) | 20 (0%) | 16 (0%) | 16 (0%) | 20 (0%) |
| threshold, mmHg | | | | | | |
| (% censored) | | | | | | |
| Median Pain threshold, | 26 (14%) | 32 (15%) | 32 (15%) | 40 (15%) | 30 (17%) | 32 (12%) |
| mmHg (% censored) | | | | | | |
| Sensory rating gas @ | 41.7 ± 2.6 | 37.2 ± 3.9 | 39.8 ± 5.0 | 32.2 ± 6.2 | 38.0 ± 5.0 | 36.0 ± 6.3 |
| 12 mmHg distension, | | | | | | |
| VAS mm | | | | | | |
| Sensory rating gas @ | 58.0 ± 2.8 | 46.3 ± 4.4 | 49.1 ± 6.0 | 40.9 ± 5.8 | 45.6 ± 5.1 | 47.2 ± 8.0 |
| 24 mmHg distension, | | | | | | |
| VAS mm | | | | | | |
| Sensory rating gas @ | 64.1 ± 2.8 | 50.6 ± 4.6 | 51.8 ± 5.8 | 48.4 ± 7.9 | 53.1 ± 5.6 | 47.2 ± 7.9 |
| 30 mmHg distension, | | | | | | |
| VAS mm | | | | | | |
| Sensory rating gas @ | 65.6 ± 3.2 | 55.9 ± 5.2 | 60.8 ± 6.4 | 46.5 ± 8.5 | 58.6 ± 6.3 | 52.3 ± 9.0 |
| 36 mmHg distension, | | | | | | |
| VAS mm | | | | | | |
| Sensory rating urgency | 45.6 ± 2.6 | 40.6 ± 3.4 | 40.9 ± 4.4 | 40.2 ± 5.6 | 42.5 ± 5.6 | 38.1 ± 5.3 |
| @ 12 mmHg distension, | | | | | | |
| VAS mm | | | | | | |
| Sensory rating urgency | 65.2 ± 2.7 | 63.3 ± 3.3 | 62.8 ± 4.4 | 64.2 ± 4.7 | 63.4 ± 4.6 | 65.2 ± 2.7 |
| @ 24 mmHg distension, | | | | | | |
| VAS mm | | | | | | |
| Sensory rating urgency | 74.6 ± 2.3 | 68.7 ± 3.3 | 67.3 ± 4.4 | 71.5 ± 4.9 | 68.8 ± 4.7 | 68.9 ± 4.6 |
| @ 30 mmHg distension, | | | | | | |
| VAS mm | | | | | | |
| Sensory rating urgency | 76.8 ± 2.5 | 76.1 ± 2.8 | 77.0 ± 3.7 | 74.2 ± 4.4 | 75.3 ± 3.9 | 77.1 ± 4.1 |
| @ 36 mmHg distension, | | | | | | |
| VAS mm | | | | | | |
| Sensory rating pain @ | 28.1 ± 2.8 | 26.4 ± 3.6 | 28.1 ± 4.4 | 23.1 + 6.3 | 27.3 ± 4.8 | 25.1 ± 5.4 |
| 12 mmHg distension, | | | | | | |
| VAS mm | | | | | | |
| Sensory rating pain @ | 46.5 ± 3.2 | 47.0 ± 4.7 | 47.6 ± 5.8 | 46.0 ± 8.1 | 45.4 ± 6.5 | 49.3 ± 6.7 |
| 24 mmHg distension, | | | | | | |
| VAS mm | | | | | | |
| Sensory rating pain @ | 55.3 ± 3.2 | 52.7 ± 4.7 | 54.9 ± 5.9 | 48.5 ± 8.2 | 50.6 ± 6.4 | 55.6 ± 7.2 |
| 30 mmHg distension, | | | | | | |
| VAS mm | | | | | | |
| Sensory rating pain @ | 57.5 ± 3.4 | 58.3 ± 4.8 | 62.2 ± 5.6 | 50.8 ± 8.8 | 57.1 ± 6.3 | 60.0 ± 7.5 |
| 36 mmHg distension, | | | | | | |
| VAS mm | | | | | | |

3.4.4 Association of Combinations of Different 3010 and 16519 Genotypes with Symptom Phenotype and Gastrointestinal Function in People with Haplogroup H

Table 13A shows the distributions of combination genotypes in patients with different FGID phenotypes, and Table 13B shows the distribution of FGID symptom phenotypes in different genotype combinations. A contingency table analysis indicated no significant association of genotype combination with symptom phenotype. In addition, no associations with gastrointestinal functions or somatic scores (Table 14) were detected. Comparisons with the AT genotype combination are constrained by the small number (n=5) with this combination of genotypes.

TABLE 13A
Proportion (%) of Different Mitochondrial DNA Genotype Combinations at
Position 3010 and 16519 in Subgroups Reporting Symptoms of Functional
GI Disorders (Chi-square p = 0.16)

| Mitochondrial DNA | | | | | | |
|---|---|---|---|---|---|---|
| Type as % of | | Chronic | Constipation | Diarrhea | | |
| Patients in each | IBS-A | Abdominal | or IBS-C | or IBS-D | Dyspepsia | Health |
| Symptom Phenotype | (n = 84) | Pain (n = 19) | (n = 155) | (n = 175) | (n = 33) | (n = 233) |
| Non-H | 64.29 | 63.16 | 63.87 | 53.14 | 51.52 | 53.22 |
| haplogroup, % | | | | | | |
| GC, % | 11.9 | 5.26 | 14.84 | 14.29 | 15.15 | 17.60 |
| GT, % | 10.71 | 5.26 | 10.32 | 13.14 | 6.06 | 14.59 |
| AC, % | 11.90 | 26.32 | 10.97 | 17.71 | 27.27 | 14.16 |
| AT, % | 1.19 | 0 | 0 | 1.71 | 0 | 0.43 |

TABLE 13B
Distribution (%) of Functional GI Disorders Based on Symptoms in H Haplogroup-
Based Genotype Combinations at Position 3010 and 16519

| | | | Chronic | | | | |
|---|---|---|---|---|---|---|---|
| Mitochondrial | | | Abdominal | | | | |
| DNA | Total n | IBS-A % | Pain % | Constipation % | Diarrhea % | Dyspepsia % | Health % |
| Non-H | 399 | 13.53 | 3.01 | 24.81 | 23.31 | 4.26 | 31.08 |
| haplogroup | | | | | | | |
| GC | 105 | 9.52 | 0.95 | 21.90 | 23.81 | 4.76 | 39.05 |
| GT | 85 | 10.59 | 1.18 | 18.82 | 27.06 | 2.35 | 40 |
| AC | 105 | 9.52 | 4.76 | 16.19 | 29.52 | 8.57 | 31.43 |
| AT | 5 | 20 | 0 | 0 | 60 | 0 | 20 |

TABLE 14
Demographic and Motor Physiological Data by 3010 (G or A) and 16519 (C or T)
Genotype Combinations (mean ± SEM)

| | GROUP: | | | |
|---|---|---|---|---|
| | GC | GT | AC | AT |
| Variable: | n = 105 | n = 85 | n = 105 | n = 5 |
| Gender, F (%) | 81 | 88 | 75 | 100 |
| BMI, kg/m² | 27.2 ± 0.5 | 27.7 ± 0.6 | 27.4 ± 0.5 | 24.2 ± 1.3 |
| Age | 43.7 ± 1.5 | 41.5 ± 1.4 | 45.6 ± 1.5 | 46.2 ± 6.7 |
| Somatic Symptom Score | 0.56 ± 0.07 | 0.54 ± 0.07 | 0.60 ± 0.07 | 0.34 ± 0.28 |
| % Stomach emptied @120 min | 49.6 ± 2.7 | 46.1 ± 2.6 | 58.2 ± 3.3 | 55.0 ± 11.1 |
| % Colonic filling @ 6 hr | 25.4 ± 6.4 | 32.9 ± 6.7 | 13.3 ± 4.3 | 16.0 ± 15.5 |
| Colon GC @ 24 hr | 2.66 ± 0.21 | 2.78 ± 0.22 | 2.95 ± 0.29 | 2.69 ± 1.02 |
| Max^{m} tolerated volume, mL | 1208 ± 106 | 1232 ± 76 | 1096 ± 85 | 1105 |
| Aggregate symptom score | 216.0 ± 17.7 | 188.1 ± 20.4 | 192.3 ± 33.1 | 152 |
| Fasting gastric volume, mL | 234.8 ± 11.5 | 257.2 ± 21.4 | 245.2 ± 12.9 | 277.3 ± 75.8 |
| Δ PP-fasting gastric vol, mL | 516.3 ± 15.8 | 509.7 ± 14.2 | 495.7 ± 17.3 | 517.7 ± 31.3 |

3.5 Discussion

Complex multifactorial conditions, usually influenced by multiple genetic and environmental factors, are the cause of the vast majority of human disease. These conditions are becoming better understood as (nuclear) genetic polymorphisms that confer an increased risk toward disease pathogenesis are rapidly being identified. Although the mitochondrial genome is small, at high copy number it constitutes up to one-third of the total cellular mass of DNA and has a very high polymorphic density (32). Thus, mtDNA sequence variation is likely to affect an individual's risk toward the development of some multifactorial conditions in a manner analogous to nuclear DNA polymorphisms (31).

The present study has shown that haplogroup H is associated with decreased odds for IBS-C and, possibly, IBS-Alt. Unlike what was reported in migraine (11), in the current study the effects of 3010A versus G are independent of the 16519 genotype. The H haplogroup was associated with increased satiation volume (relative to all non-H haplogroups). Within haplogroup H, decreased gastric emptying at 120 minutes was found with the 3010A genotype (relative to the 3010G genotype). The significance of these observations is unclear and requires replication in an independent cohort of patients. A post-hoc analysis suggested an association between 3010A genotype and a non-specific abdominal pain group created by combining chronic abdominal pain and dyspepsia. This also requires confirmation in a separate cohort. Interestingly, it is known that accelerated gastric emptying is one of the factors that contribute to development of dyspepsia (33), although, in our participants, both pain and slower gastric emptying were associated with 3010A. The increased maximum tolerated volume associated with H haplogroup overall may also increase upper gastrointestinal symptoms. Epidemiological data from patients with obesity in a U.S. community showed a significant increase in the reporting of abdominal pain (34). Furthermore, the present association of 3010A with non-specific abdominal pain is consistent with the Boles group's previously reported association of this SNP with both migraine and cyclic vomiting syndrome (35) conditions in which abdominal pain/discomfort is common. While prior work has reported an association between mitochondrial dysfunction (as measured by ATP production rate in biopsied muscle) and the presence of somatic symptoms in general (36, 37), in the current study we found no association between the 3 mtDNA SNPs studied and the overall somatic symptom score. Further research is needed to better understand these findings

While haplogroup H is defined by the 7028C mtDNA SNP, it is unknown which mtDNA polymorphism(s) actually confers the functional consequence, as haplogroup H is highly complex with multiple constituent sub-haplogroupings. On the other hand, in our previous study, 16519T and 3010A were considered highly likely to confer the functional consequences in cyclic vomiting syndrome, as the full mtDNA genomic sequences in those subjects were available for analysis. This led to the current study to explore whether these SNPs are also factors associated with IBS.

The 16519 SNP is located in the 1-kb noncoding mtDNA control region (“D-loop”), while the 3010 SNP is located in the 16S ribosomal RNA gene. Both polymorphisms are located in areas with relatively low sequence heterogeneity, have arisen multiple times in human evolution, are present in all major human races, and have been reported to likely have functional consequences (11, 31). 16519T is associated with diabetes and a poorer prognosis in individuals with pancreatic cancer (38), and a physiological effect of this polymorphism is also suggested by its complex effects on human exercise physiology (39). 3010A defines two East-Asian sub-haplogroups, J1 and D4, which are overrepresented in centenarians (40), but 3010A by itself has not previously been found to be statistically associated with human disease other than cyclic vomiting syndrome and migraine (11). The present data suggest that 3010A may be associated with slower gastric emptying at 120 minutes compared to the 3010G genotype; whether this might explain, at least in part, the development of vomiting in cyclic vomiting syndrome and migraine is unclear.

There are several points of caution in the interpretation of our study. First is the relatively small sample size for genotype to symptom phenotype associations. We chose to sacrifice a larger number of subjects for substantially greater genotypic homogeneity by limiting this study to subjects of haplogroup H. On the other hand, we have included unique and validated measures of gastrointestinal motor and sensory functions to explore possible hypotheses regarding the association between mtDNA and gastrointestinal functions. Thus, the sample size in the study is generally appropriate for the study of associations between the mtDNA genotype and haplogroups (with their documented prevalence in the community) and the motor, satiation and sensory functions.

Second, the functional effects of the two SNPs at position 3010 and 16519 in a model cell or reporter system have not been demonstrated and, therefore, even if there is an association, the mechanism whereby dysfunction or symptoms occur is unclear. It is also conceivable that 3010 and 16519 may serve simply as markers that are in linkage disequilibrium with an etiologically significant genetic variant, if such a relationship truly exists (e.g., between 3010 and altered gastric emptying).

Third, one should consider whether familial aggregation studies unequivocally support a component of maternal inheritance, since an unequivocal maternal pattern of inheritance would support the potential role of mtDNA in the inherited component of IBS. It is worth noting, therefore, that although the odds ratios for female transmission in IBS with familial aggregation is significant, whereas that for males and brothers is not (2), the sample size for men in the previously published paper (2) was small (˜18% of cohort), the confidence intervals wide (OR 4.2, CI 0.8, 21.0) and a type II error cannot be excluded. Thus, familial aggregation studies do not exclude a non-maternal inheritable component in IBS.

We conclude that the Western Eurasian mtDNA haplogroup H (7028 genotype) is associated with decreased odds ratios for IBS-C and, possibly, IBS-Alt. The H haplogroup may also influence satiation. Within haplogroup H, the common mtDNA polymorphism, 3010A, is associated with non-specific abdominal pain and delayed gastric emptying at 120 minutes.

These disturbances of gastric function may be relevant in disorders of satiation (e.g., anorexia nervosa or obesity) and gastric emptying (e.g., gastroparesis and functional dyspepsia). Although only haplogroup H was studied, 16519T and 3010A are found in individuals with a multitude of haplogroups and within all major races. In fact, among Americans of European origin, 16519T is actually slightly more common among non-haplogroup H individuals (41). It is unclear whether the effect of these polymorphisms may be dependent upon the background haplogroup, and further study is needed. Further studies are also required to assess whether the significant associations with pain and processes associated with satiation result directly from functional effects of the polymorphisms on gastrointestinal motor and sensory functions. However, as recommended by Mayer (42), investigation of the endophenotype provides the first evidence that reported associations of mtDNA with functional syndromes such as cyclic vomiting syndrome and migraine have an identified physiological basis. 3.6 References
1. Kalantar J S, Locke G R 3rd, Zinsmeister A R, Beighley C M, Talley N J. Familial aggregation of irritable bowel syndrome: a prospective study. Gut 2003; 52:1703-1707.
2. Saito Y A, Zimmerman J M, Harmsen W S, De Andrade M, Locke GR 3rd, Petersen G M, Talley N J. Irritable bowel syndrome aggregates strongly in families: a family-based case-control study. Neurogastroenterol Motil 2008; 20:790-797.
3. Van Kerkhoven L A, Laheij R J, Jansen J B. Meta-analysis: a functional polymorphism in the gene encoding for activity of the serotonin transporter protein is not associated with the irritable bowel syndrome. Aliment Pharmacol Ther 2007; 26; 979-986.
4. Holtmann G, Siffert W, Haag S, Mueller N, Langkafel M, Senf W, Zotz R, Talley N J. G-protein beta 3 subunit 825 CC genotype is associated with unexplained (functional) dyspepsia. Gastroenterology 2004; 126:971-979.
5. Camilleri C E, Carlson P J, Camilleri M, Castillo E J, Locke G R 3rd, Geno D M, Stephens D A, Zinsmeister A R, Urrutia R. A study of candidate genotypes associated with dyspepsia in a U.S. community. Am J Gastroenterol 2006; 101:581-592.
6. Tahara T, Arisawa T, Shibata T, Wang F, Nakamura M, Sakata M, Hirata I, Nakano H. Homozygous 825T allele of the GNB3 protein influences the susceptibility of Japanese to dyspepsia. Dig Dis Sci 2008; 53:642-646.
7. van Lelyveld N, Linde J T, Schipper M, Samsom M. Candidate genotypes associated with functional dyspepsia. Neurogastroenterol Motil 2008; 20:767-773.
8. Camilleri M. Mechanisms in IBS: something old, something new, something borrowed. Neurogastroenterol Motil 2005; 17:311-316.
9. Johns D R. Seminars in medicine of the Beth Israel Hospital, Boston. Mitochondrial DNA and disease. N Engl J Med 1995; 333:638-644.
10. Taylor R W, Turnbull D M. Mitochondrial DNA mutations in human disease. Nat Rev Genet 2005; 6:389-402.
11. Boles R G, Adams K, Li B U. Maternal inheritance in cyclic vomiting syndrome. Am J Med Genet 2005; 133:71-77.
12. Zaki E A, Freilinger T, Klopstock T, Baldwin E E, Heisner K R U, Adams K, Dichgans M, Boles R G. Two common mitochondrial DNA polymorphisms are highly associated with migraine headache and cyclic vomiting syndrome (submitted).
13. Boles R G, Chun N, Senadheera D, Wong L J. Cyclic vomiting syndrome and mitochondrial DNA mutations. Lancet 1997; 350:1299-1300.
14. Ito M, Le S T, Chaudhari D, Higashimoto T, Maslim A, Boles R G. Screening for mitochondrial DNA heteroplasmy in children at risk for mitochondrial disease. Mitochondrion 2001; 1:269-278.
15. Wang Q, Ito M, Adams K, Li B U, Klopstock T, Maslim A, Higashimoto T, Herzog J, Boles R G. Mitochondrial DNA control region sequence variation in migraine headache and cyclic vomiting syndrome. Am J Med Genet 2004; 131:A50-58.
16. Burnett B B, Gardner A, Boles R G. Mitochondrial inheritance in depression, dysmotility and migraine? J Affect Disord 2005; 88: 109-116.
17. Wong L-J C, Boles R G. Mitochondrial DNA analysis in clinical laboratory diagnostics. Clin Chim Acta 2005; 354:1-20.
18. Talley N J, Phillips S F, Wiltgen C M, Zinsmeister A R, Melton L J 3rd. Assessment of functional gastrointestinal disease: the bowel disease questionnaire. Mayo Clin Proc 1990; 65:1456-1479.
19. Andresen V, Camilleri M, Kim H J, Stephens D A, Carlson P J, Talley N J, Saito Y A, Urrutia R, Zinsmeister A R. Is there an association between GNβ3 C825T genotype and lower functional gastrointestinal disorders? Gastroenterology 2006; 130:1985-1994.
20. Castillo E J, Camilleri M, Locke G R III, Burton D D, Stephens D A, Geno D M, Zinsmeister A R. A community based, controlled study of the epidemiology and pathophysiology of dyspepsia. Clin Gastroenterol Hepatol 2004; 2:985-996.
21. Kim H J, Camilleri M, Carlson P J, Cremonini F, Ferber I, Stephens D, McKinzie S, Zinsmeister A R, Urrutia R. Association of distinct U2 adrenoceptor and serotonin-transporter polymorphisms associated with constipation and somatic symptoms in functional gastrointestinal disorders. Gut 2004; 53:829-837.
22. Chial H J, Camilleri C, Delgado-Aros S, Burton D, Thomforde G, Ferber I, Camilleri M. A nutrient drink test to assess maximum tolerated volume and postprandial symptoms: effects of gender, body mass index and age in health. Neurogastroenterol Motil 2002; 14:249-253.
23. Camilleri M, Atanasova E, Carlson P J, Ahmad U, Kim H J, Viramontes B E, McKinzie S, Urrutia R. Serotonin-transporter polymorphism pharmacogenetics in diarrhea-predominant irritable bowel syndrome. Gastroenterology 2002; 123:425-432.
24. Camilleri M, Busciglio I, Carlson P, McKinzie S, Burton D, Baxter K, Ryks M, Zinsmeister A R. Pharmacodynamics and pharmacogenetics of low dose clonidine in irritable bowel syndrome. Neurogastroenterol Motility (submitted).
25. Cremonini F, Mullan B P, Camilleri M, Burton D D, Rank M R. Performance characteristics of scintigraphic transit measurements for studies of experimental therapies. Aliment Pharmacol Ther 2002; 16:1781-1790.
26. Bouras E P, Delgado-Aros S, Camilleri M, Castillo E J, Burton D D, Thomforde G M, Chial H J. SPECT imaging of the stomach: comparison with barostat, and effects of sex, age, body mass index, and fundoplication. Single photon emission computed tomography. Gut 2002; 51:781-786.
27. Camilleri M, McKinzie S, Busciglio I, Low P A, Sweetser S, Burton D, Baxter K, Ryks M, Zinsmeister A R. Prospective study of motor, sensory, psychological and autonomic functions in patients with irritable bowel syndrome. Clin Gastroenterol Hepatol 2008; 6:772-781.
28. Hammer H F, Phillips S F, Camilleri M, Hanson R B. Rectal tone, distensibility, and perception: reproducibility and response to different distensions. Am J Physiol 1998; 274:G584-G590.
29. Cremonini F, Houghton L A, Camilleri M, Ferber I, Fell C, Cox V, Castillo E J, Alpers DH, Dewit O E, Gray E, Lea R, Zinsmeister A R, Whorwell P J. Barostat testing of rectal sensation and compliance in humans: comparison of results across two centres and overall reproducibility. Neurogastroenterol Motil 2005; 17:810-820.
30. Wallace D C, Brown M D, Lott M T. Mitochondrial DNA variation in human evolution and disease. Gene 1999; 238:211-230.
31. Ruiz-Pesini E, Lott M T, Procaccio V, Poole J, Brandon M C, Mishmar D, et al. An enhanced MITOMAP with a global mtDNA mutational phylogeny. Nucleic Acids Research 2007; 35:D823-D828.
32. Shoffner J M, Wallace D C. Oxidative phosphorylation diseases and mitochondrial DNA mutations: diagnosis and treatment. Annu Rev Nutr 1994; 14:535-568.
33. Delgado-Aros S, Camilleri M, Cremonini F, Ferber I, Stephens D, Burton D D. Contributions of gastric volumes and gastric emptying to meal size and post-meal symptoms in functional dyspepsia. Gastroenterology 2004; 127:1685-1694.
34. Delgado-Aros S, Locke G R III, Camilleri M, Talley N J, Fett S, Zinsmeister A R, Melton L J III. Obesity is associated with increased risk of gastrointestinal symptoms: a population-based study. Am J Gastroenterol 2004; 99:1801-1806.
35. Boles R, Gardner A. Sex ratios and mitochondrial genetics in migraine. Cephalalgia. 2008 Jul. 15. [Epub ahead of print].
36. Gardner A, Boles R G. Mitochondrial energy depletion in depression with somatization. Psychother Psychosom. 2008; 77(2):127-9.
37. Gardner A, Boles R G. Symptoms of somatization as a rapid screening tool for mitochondrial dysfunction in depression. Biopsychosoc Med. 2008 Feb. 22; 2:7].
38. Navaglia F, Basso D, Fogar P, Sperti C, Greco E, Zambon C F, et al. Mitochondrial DNA D-loop in pancreatic cancer: somatic mutations are epiphenomena while the germline 16519 T variant worsens metabolism and outcome. Am J Clin Pathol 2006; 126:593-601.
39. Murakami H, Ota A, Simojo H, Okada M, Ajisaka R, Kuno S. Polymorphisms in control region of mtDNA relates to individual differences in endurance capacity or trainability. Japan J Physiol 2002; 52:247-256.
40. Rose G, Passarino G, Carrieri G, Altomare K, Greco V, Bertolini S, et al. Paradoxes in longevity: sequence analysis of mtDNA haplogroup J in centenarians. Eur J Hum Genet 2001; 9:701-707.
41. Coble M D, Just R S, O'Callaghan J E, Letmanyi I H, Peterson C T, Irwin J A, Parsons T J. Single nucleotide polymorphisms over the entire mtDNA genome that increase the power of forensic testing in Caucasians. Int J Legal Med 2004; 118:137-146.
42. Mayer E A. The challenge of studying the biology of complex, symptom-based GI disorders. Gastroenterology. 2008; 134: 1826-7.

### Example 4

### Mitochondrial DNA as a Genetic Factor in SIDS

4.1 Introduction

Despite the success of the “Back to Sleep” campaign, in industrialized societies the sudden infant death syndrome (SIDS) remains the primary cause of infant mortality for infants one to twelve months of age. While remaining essentially idiopathic, many authors have demonstrated abnormal autonomic responses in SIDS. The unique age distribution, peaking at age 2 to 4 months, coincides with the stage that infants generally start to sleep throughout the night, a potential clue known to tired parents worldwide. In addition, SIDS is frequently temporally associated with viral infections (1), which typically reduce oral intake. Thus, we hypothesized that infants with underlying genetic polymorphisms predisposing towards hypoglycemia and/or anomalous autonomic responses succumb when hepatic glycogen (energy) stores are first depleted. In support of our hypothesis, autopsy data in 254 SIDS cases revealed that 20% had marked hepatic glucose depletion on autopsy (2), suggesting the presence of terminal hypoglycemia. Fully 92% of those infants succumbed between the ages of 1 to 4.9 months (2).

Recently, we described two common maternally-inherited mitochondrial DNA (mtDNA) polymorphisms, 16519T and 3010A, which are strongly and significantly associated with migraine and cyclic vomiting syndrome (odds ratios of 3-17) (3), both of which are functional disorders with prominent dysautonomic features. Since fasting hypoglycemia and SIDS are relatively common among the matrilineal relatives in families demonstrating the maternal inheritance of functional/dysautonomic conditions, and since mtDNA sequence variation in SIDS has been described (4), we hypothesized that these two mtDNA polymorphisms constitute part of the genetic susceptibility towards SIDS.

4.2 Methods

The 16519 and 3010 polymorphisms were assayed by PCR/RFLP as previously described (3) in the SIDS subjects from the above study (2, 5). This study was restricted to haplogroup H in order to increase study power, because of its relative mtDNA sequence homogeneity (3). Haplogroup H includes about 40-50% of individuals of Northern European heritage. Cases previously reported as likely to have a fatty acid oxidation disorder (5) were excluded. Postmortem hepatic glucose concentrations were previously determined in the above study (2, 5).

4.3 Results

The “glucose-depleted” SIDS subgroup is highly enriched with the 3010G+16519T genotype (“GT”, 8 subjects with GT versus 1 with AC), while the “glucose-normal” SIDS subgroup is highly enriched with the 3010A+16519C genotype (“AC”, 1 subject with GT versus 10 with AC).

4.4 Discussion

Our data suggests pathophysiological heterogeneity in SIDS, in particular supporting the existence of a subgroup (approximately 20% of cases) with hepatic glucose depletion (probable terminal hypoglycemia), which is associated with the GT mtDNA genotype. Thus, through mtDNA testing, it may be possible to prospectively identify a group of infants at high risk for hypoglycemia during fasting and/or high metabolic demands (i.e., fever). For those susceptible infants, the simple intervention of preventing fasting by frequent feedings, especially during viral infections, may prevent hypoglycemia, which might otherwise progress to SIDS.

Furthermore, the AC mtDNA genotype is highly associated with the majority (“glucose normal”) subgroup of SIDS. This association is mostly due to the 3010A polymorphism, which appears to more than triple the odds that an infant will succumb to this dominant type of SIDS (relative to controls:P=0.007, odds ratio 3.5, 95% C.I. 1.3-9.1). This common polymorphism in the 16S-ribosomal RNA gene is not “neutral”, as it is associated with migraine on a 16519T background (3) and is protective against stroke (6). The 3010A polymorphism affects ribosomal RNA secondary structure, suggesting a possible effect through mitochondrial translation. Thus, our pilot data suggest that some of the genetic component predisposing towards SIDS is maternally inherited on the mtDNA, and further research is needed.

4.5 References
1. Samuels M. Viruses and sudden infant death. Paediatr Respir Rev. 2003; 4(3):178-183.
2. Boles R G, Rinaldo R. Glucose concentration in 254 SIDS livers suggests pathophysiological heterogeneity. Pediatr and Dev Pathol. 2006; 9(1):86-87.
3. Zaki E A, Freilinger T, Klopstock T, et al. Two common mitochondrial DNA polymorphisms are highly associated with migraine headache and cyclic vomiting syndrome. Cephalalgia In Press.
4. Arnestad M, Opdal S H, Musse M A, Vege A, Rognum TO. Are substitutions in the first hypervariable region of the mitochondrial DNA displacement-loop in sudden infant death syndrome due to maternal inheritance? Acta Paediatr. 2002; 91(10): 1060-1064.
5. Boles R G, Buck E A, Blitzer M G, et al. Retrospective biochemical screening of fatty acid oxidation disorders in postmortem liver of 418 cases of sudden death in the first year of life. J Pediatr. 1998; 132(6):924-933.
6. Rosa A, Fonseca B V, Krug T, et al. Mitochondrial haplogroup Hi is protective for ischemic stroke in Portuguese patients. BMC Med Genet. 2008; 9:57.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety for any purpose.

1. A method to diagnose a functional disorder or functional symptomatology in a human comprising:
determining the presence of a polymorphism in mitochondrial DNA of the human, wherein the polymorphism is selected from the group consisting of 239C, 2259T, 3010A, 3010G, 4727G, 4745G, 7337A, 9380A, 13326C, 13680T, 14831 A, 14872T, 16519T, and 16519C; and
identifying the human as being at an increased risk to suffer from a functional disorder when compared to a human without said polymorphism.

2. The method according to claim 1, said method comprising determining the presence of at least two of said polymorphisms selected from the group consisting of 239C, 2259T, 3010A, 3010G, 4727G, 4745G, 7337A, 9380A, 13326C, 13680T, 14831A, 14872T, 16519T, and 16519C.

3. The method according to claim 1, said method comprising determining the presence of at least three of said polymorphisms selected from the group consisting of 239C, 2259T, 3010A, 3010G, 4727G, 4745G, 7337A, 9380A, 13326C, 13680T, 14831A, 14872T, 16519T, and 16519C.

4. The method according to claim 1, said method comprising determining the presence of at least four of said polymorphisms selected from the group consisting of 239C, 2259T, 3010A, 3010G, 4727G, 4745G, 7337A, 9380A, 13326C, 13680T, 14831A, 14872T, 16519T, and 16519C.

5. The method according to claim 1, said method comprising determining the presence of at least five of said polymorphisms selected from the group consisting of 239C, 2259T, 3010A, 3010G, 4727G, 4745G, 7337A, 9380A, 13326C, 13680T, 14831A, 14872T, 16519T, and 16519C.

6. The method according to claim 1, said method comprising determining the presence of a polymorphisms selected from the group consisting of 3010A, 3010G, 16519T, and 16519C.

7. The method according to claim 6, said method comprising determining the presence of at least two of polymorphisms selected from the group consisting of 3010A, 3010G, 16519T, and 16519C.

8. The method according to claim 6, said method comprising determining the presence of at least three of said polymorphisms selected from the group consisting of 3010A, 3010G, 16519T, and 16519C.

9. The method according to claim 6, said method comprising determining the presence of four of said polymorphisms selected from the group consisting of 3010A, 3010G, 16519T, and 16519C.

10. The method according to claim 1, wherein said functional disorder is selected from the group consisting of chronic fatigue syndrome (CFS), migraine, irritable bowel syndrome (IBS), depression, fibromyalgia, complex regional pain syndrome, nonspecific abdominal pain, chronic temporal mandibular joint pain, myalgic encephalitis, chronic pelvic pain, chronic pain syndromes, interstitial urethritis, post-traumatic stress syndrome, gulf war syndrome, functional tinnitus, sudden infant death syndrome (SIDS), and hypoglycemia.

11. The method according to claim 1, wherein said functional symptomatology is selected from the group consisting of gastrointestinal dysmotility, gas, pain, migraine, cyclic vomiting, chronic fatigue, limb pain, constipation, diarrhea, sleep apnea, frequent urination, and gastroesophageal reflux.

12. The method according to claim 6, wherein said functional disorder is selected from the group consisting of chronic fatigue syndrome (CFS), migraine, irritable bowel syndrome (IBS), depression, fibromyalgia, complex regional pain syndrome, nonspecific abdominal pain, chronic temporal mandibular joint pain, myalgic encephalitis, chronic pelvic pain, chronic pain syndromes, interstitial urethritis, post-traumatic stress syndrome, gulf war syndrome, functional tinnitus, sudden infant death syndrome (SIDS), and hypoglycemia.

13. The method according to claim 6, wherein said functional symptomatology is selected from the group consisting of gastrointestinal dysmotility, gas, pain, migraine, cyclic vomiting, chronic fatigue, limb pain, constipation, diarrhea, sleep apnea, frequent urination, and gastroesophageal reflux.

14. A kit for diagnosing a functional disorder in a human comprising two oligonucleotides capable of hybridizing to human mitochondrial DNA 5′ and 3′ to a nucleotide in the DNA, wherein the nucleotide is selected from the group consisting of 239, 2259, 3010, 4727, 4745, 7337, 9380, 13326, 13680, 14831, 14872, and/or 16519; and wherein said oligonucleotides are separated by 0 to 200 nucleotides; and further comprising instructions for using the kit to diagnose functional disorders.

15. A kit for diagnosing a functional disorder in a human comprising means for detecting the presence of a polymorphism in mitochondrial DNA of the human, wherein the polymorphism is selected from the group consisting of 239C, 2259T, 3010A, 3010G, 4727G, 4745G, 7337A, 9380A, 13326C, 13680T, 14831A, 14872T, 16519T, and 16519C; and further comprising instructions for using the kit to diagnose functional disorders.
